(19) United States
(12) Patent Application Publication
(10) Pub. No.: US 2024/0336880 A1
(43) Pub. Date: Oct. 10, 2024

(54) BIODEGRADABLE PREPOLYMER MICROCAPSULES

(71) Applicant: INTERNATIONAL FLAVORS & FRAGRANCES INC., NEW YORK, NY (US)

(72) Inventors: Takashi SASAKI, Morganville, NJ (US); Jason HACKENBERG, Aberdeen, NJ (US); Ryan CHIN, Matawan, NJ (US); Lewis Michael POPPLEWELL, Union Beach, NJ (US); Ronald GABBARD, Farmingdale, NJ (US); Yabin LEI, Holmdel, NJ (US); Sorin Nicolae SAUCA, Timisoara (RO); Timothy YOUNG, Middletown, NJ (US); Tram TRAN, New Brunswick, NJ (US); Helen LU, Wallingford, NJ (US); Min ZHAO, Audobon, PA (US); Julie Ann WIELAND, Edison, NJ (US)

(21) Appl. No.: 18/724,124

(22) PCT Filed: Jan. 12, 2023
(86) PCT No.: PCT/US2022/010696

§ 371 Date: Jun. 25, 2024

(30) Foreign Application Priority Data
Jan. 14, 2022 (EP) 22151570.3

(51) Int. Cl. C11D 3/50; B01J 13/16; C11D 3/00; C11D 3/37; C11D 11/00; C11D 17/00

(52) U.S. Cl. CPC C11D 3/505; B01J 13/16; C11D 3/0015; C11D 3/3726; C11D 11/0082; C11D 17/0039

(57) ABSTRACT

This disclosure relates to a biodegradable core-shell microcapsule that includes (a) a shell containing the reaction product of an isocyanate-functionalized prepolymer with a crosslinker and optionally a polyelectrolyte under an aqueous condition, and (b) a core containing an active material, wherein the isocyanate-functionalized prepolymer is the reaction product of a polyisocyanate with a biopolymer and/or an amphiphilic compound under an anhydrous condition, and the microcapsule shell is substantially free of or free of a self-condensed polyisocyanate. This disclosure also relates to a process of preparing such biodegradable core-shell microcapsule. This disclosure also relates to a consumer product containing such biodegradable core-shell microcapsule. This disclosure also relates to a process of making the isocyanate-functionalized prepolymer.

### FIELD OF THE DISCLOSURE

The present disclosure relates to biodegradable prepolymer microcapsules, as well as their use in consumer products and a process for preparing the biodegradable prepolymer microcapsules.

### BACKGROUND OF THE DISCLOSURE

Current commercial fragrance capsules including melamine-formaldehyde and polyurea are made from synthetic materials, which are not readily biodegradable. This is an issue for several reasons. Firstly, consumers are demanding more environmentally friendly products. Secondly, due to the potential for new regulations, for example from the European Chemicals Agency (ECHA), there may be a ban on the use of microplastics in a variety of consumer goods products (e.g., cosmetics, detergents, etc.). As a result, there is an ever-increasing demand for fragrance delivery technologies where the capsule wall material is more biodegradable and/or sustainable, and will satisfy the requirements set out in any currently existing and newly proposed regulations banning the use of microplastics.

While methods to include biopolymer composite materials in the wall of microcapsules have been described, polyisocyanates are first dissolved in the oil phase and then emulsified together with an aqueous phase containing polyamines, polyelectrolytes or biopolymers to react at the oil-water interface. See U.S. Pat. No. 8,663,690 B2 (Rice University); U.S. Pat. No. 10,034,819 B2 (Firmenich); EP 3478403 A1 (Firmenich); GB 2029791 A (Oji Paper); US 2021/0339217 A1 (Encapsys Inc.); U.S. Pat. No. 9,770,608 B2 (International Flavors & Fragrances Inc.); WO 2020/209907 A1 (International Flavors & Fragrances Inc.); WO 2020/209908 A1 (International Flavors & Fragrances Inc.); WO 2020/209909 A1 (International Flavors & Fragrances Inc.); WO 2020/194910 A1 (Fujifilm); WO 2020/195132 A1 (Fujifilm); WO 2021/116365 A1 (Henkel); WO 2021/115601 A1 (Koehler SE), WO 2021/116306 A1 (Firmenich); WO 2021/122636 A1 (Firmenich); WO 2021/122630 A1 (Firmenich); and WO 2021/122633 A1 (Firmenich). While the intended reaction is an interfacial polymerization, controlling the kinetics to yield a copolymer/composite is challenging and often results in self-condensation of polyisocyanate coated with a layer of unreacted material originally intended for the interfacial polymerization. However, the degree or rate of biodegradability of these capsules is not always described.

Even assuming, arguendo, that these microcapsules described above may pass any current biodegradation tests (e.g., the OECD301F or OECD310), they may still not be “truly” biodegradable and fully ECHA compliant. By that we mean that the microcapsules may not satisfy the requirements under the proposed ECHA regulations that materials forming the microcapsule wall cannot be a “blend” of biodegradable materials (e.g., biopolymers) and non-biodegradable materials. Alternatively, if the microcapsule wall is considered a blend, all components of the blend as a whole need to meet the requirements for biodegradability (e.g., minimally at least the OECD301F or OECD310 tests).

Polyurea/polyurethane co-polymers and composites prepared from the reaction of polyisocyanates and biodegradable polyelectrolytes/biopolymers such as gelatin and chitosan have been described in terms of mechanical properties and functional benefits (Bertoldo et al. (2007) Macromol. Biosci. 7:328-338; Gallego (2013) Molecules 18:6532-6549; Koebel et al. (2016) J. Sol-Gel Sci. Technol. 79:308-318). In addition, nanoparticle composite materials having a matrix composed of chitosan and tannins have been described for drug delivery (U.S. Pat. No. 10,104,888 B2, Wisconsin Alumni Research Foundation). However, use of these materials to deliver a fragrance via consumer products has not been described.

Accordingly, microcapsules composed of sustainable, and/or biodegradable materials, which are chemically stable and/or deliver active materials such as fragrances in consumer products are still needed in the industry. Preferably, the materials used to form the microcapsule walls do not form a blend of biodegradable materials and non-biodegradable materials. Alternatively, if the materials used to form the microcapsule walls do form a blend, the blend contains only a small amount (i.e., ≤10%, ≤5%, ≤3%, ≤1%, ≤0.5%, ≤0.1% or ≤0.05%) of non-biodegradable materials, or all components of the blend as a whole meet the requirements for biodegradability. The microcapsules preferably also provide acceptable fragrance profile and performance in use. The biodegradable core-shell microcapsules of the present disclosure satisfy this and other needs in the industry.

### BRIEF SUMMARY OF THE DISCLOSURE

The biodegradable core-shell microcapsule of the present disclosure is based, inter alia, on the discovery that biodegradable and/or sustainable microcapsule shells can be prepared with isocyanate-functionalized prepolymers.

Accordingly, the present disclosure provides a biodegradable core-shell microcapsule comprising: (a) a microcapsule shell comprising the reaction product of an isocyanate-functionalized prepolymer with a crosslinker and optionally a polyelectrolyte under an aqueous condition; and (b) a microcapsule core comprising an active material; wherein the isocyanate-functionalized prepolymer is the reaction product of a polyisocyanate with a biopolymer and/or an amphiphilic compound under an anhydrous condition, the biopolymer is selected from the group consisting of gelatin, collagen, chitosan, modified guar, modified glucan, gum Arabic (gum acacia), modified gum Arabic (modified gum acacia), proteins (e.g., pea protein), hydrolyzed proteins (e.g., hydrolyzed pea protein), fermented proteins, hydrophobin, enzymes, alginate, carrageenan, pectin, modified starch, modified cellulose, and mixtures thereof, the amphiphilic compound is selected from the group consisting of partially neutralized acid esters, polyvinyl alcohol, glycolipids, fatty acids, saponins, quillaia extract, surfactant salts having carboxylate and/or linear alcohol groups, and mixtures thereof, the microcapsule shell has a biodegradation rate of at least 20%, 30%, 40%, 50%, 60%, 65%, 70%, 75%, 80%, 85%, 90%, 95% or 98%, based on the weight of the microcapsule shell, within 60 days according to OECD301F, and the microcapsule shell is substantially free of or free of a self-condensed polyisocyanate. In some embodiments, the partially neutralized acid ester is a partially neutralized citric acid ester.

The present disclosure also provides a process for preparing a biodegradable core-shell microcapsule. The process comprises (a) reacting, under an anhydrous condition, a polyisocyanate with a biopolymer and/or an amphiphilic compound, preferably in the presence of a catalyst, to form an isocyanate-functionalized prepolymer, preferably the polyisocyanate is dissolved in a solution comprising a solvent and/or an active material; (b) emulsifying the isocyanate-functionalized prepolymer with an aqueous solution to form an emulsion, preferably said aqueous solution comprises a polyelectrolyte; (c) crosslinking the isocyanate-functionalized prepolymer and optionally the polyelectrolyte with a first crosslinker to form the biodegradable core-shell microcapsule, wherein the first crosslinker comprises an oxidized sugar comprising aldehyde groups and/or an enzyme selected from the group consisting of transglutaminase, laccase, peroxidase, oxidase, amylase, transferase, and mixtures thereof; (d) optionally further crosslinking the microcapsule shell with a second crosslinker selected from the group consisting of tannic acid, hydrolyzed tannic acid, tannin, gallic acid, methyl gallate, ethyl gallate, glutaraldehyde, glyoxal, triethyl citrate, malondialdehyde, genipin, dopamine, phenols, polyphenols, polycarbodiimide, polyacid chlorides, tetraethoxysilane, enzymes, multivalent cations, and mixtures thereof; and (e) optionally curing the microcapsule shell at a temperature ranging from 5° C. to 150° C. and at a pH ranging from 2 to 11; wherein the microcapsule shell has a biodegradation rate of at least 20%, 30%, 40%, 50%, 60%, 65%, 70%, 75%, 80%, 85%, 90%, 95% or 98%, based on the weight of the microcapsule shell, within 60 days according to OECD301F, and the microcapsule shell is substantially free of or free of a self-condensed polyisocyanate. The present disclosure also provides a biodegradable core-shell microcapsule obtainable by such process.

The present disclosure also provides a process for making an isocyanate-functionalized prepolymer. The process comprises reacting, under an anhydrous condition, a polyisocyanate with a biopolymer and/or an amphiphilic compound, preferably in the presence of a catalyst, to form an isocyanate-functionalized prepolymer, wherein the polyisocyanate is dissolved in a solution comprising a solvent and/or an active material, the biopolymer is selected from the group consisting of gelatin, collagen, chitosan, modified guar, modified glucan, gum Arabic (gum acacia), modified gum Arabic (modified gum acacia), proteins (e.g., pea protein), hydrolyzed proteins (e.g., hydrolyzed pea protein), fermented proteins, hydrophobin, enzymes, alginate, carrageenan, pectin, modified starch, modified cellulose, and mixtures thereof, and the amphiphilic compound is selected from the group consisting of partially neutralized acid esters, polyvinyl alcohol, glycolipids, fatty acids, saponins, quillaia extract, surfactant salts having carboxylate and/or linear alcohol groups, and mixtures thereof. In some embodiments, the partially neutralized acid ester is a partially neutralized citric acid ester.

### DETAILED DESCRIPTION

The foregoing general description and the following detailed description are exemplary and explanatory only and are not restrictive of the invention, as defined in the appended claims. Other features and benefits of any one or more of the embodiments will be apparent from the following detailed description, and from the claims.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. For example, when a range of “1 to 10” is recited, the recited range should be construed as including ranges “1 to 8”, “3 to 10”, “2 to 7”, “1.5 to 6”, “3.4 to 7.8”, “1 to 2 and 7-10”, “2 to 4 and 6 to 9”, “1 to 3.6 and 7.2 to 8.9”, “1-5 and 10”, “2 and 8 to 10”, “1.5-4 and 8”, and the like.

The present disclosure illustratively described herein suitably may be practiced in the absence of any element or elements, limitation or limitations that are not specifically disclosed herein. While compositions and methods are described herein in terms of “comprising” various components or steps, the compositions and methods also can “consist essentially of” or “consist of” the various components or steps, unless stated otherwise.

All parts, percentages and proportions referred to herein and in the claims are by weight unless otherwise indicated.

A person of ordinary skill in the art appreciates that some proteins can be modified, denatured and/or hydrolyzed. Unless explicitly indicated, a protein in this disclosure includes its modified, denatured and/or hydrolyzed forms. A person of ordinary skill in the art also appreciates that some biopolymers and polyelectrolytes can be modified and/or hydrolyzed. Unless explicitly indicated, a biopolymer or a polyelectrolyte in this disclosure includes its modified and/or hydrolyzed forms.

Before addressing details of embodiments described below, some terms are defined or clarified.

The term “elevated temperature”, as used herein, means a temperature higher than the room temperature.

The terms “obtainable” and “obtained” can be used interchangeably in this disclosure and do not mean to indicate that, e.g., a product must be obtained by, e.g., the sequence of steps following the term “obtained” though such a limited understanding is always included by the terms as a preferred aspect of the present disclosure.

The term “microcapsule slurry”, as used herein, means an aqueous suspension of the biodegradable core-shell microcapsule. In some embodiments, the biodegradable core-shell microcapsule product produced in accordance with the methods and examples described in the present disclosure is in the form of a microcapsule slurry. The microcapsule slurry may be used directly in a consumer product. The microcapsule slurry may also be washed, coated, dried (e.g., spray-dried) and/or combined with one or more other microcapsules, active materials, and/or carrier materials.

The term “self-condensed polyisocyanate”, as used herein, means the polyurea formed by the self-polymerization of polyisocyanate in the presence of water. A person skilled in the art appreciates that isocyanate can react with water to form amine which can further react with isocyanate to form urea linkage. Accordingly, polyisocyanate can self-polymerize in the presence of water to form polyurea. The self-condensed polyisocyanate is non-biodegradable.

The term “under an aqueous condition”, as used herein with respect to a reaction, means the reaction is conducted in the substantial presence of water. In some embodiments, it means the reaction is conducted in the presence of an aqueous phase. In some embodiments, it means the water content in the reaction mixture (including reactants, products, solvents, catalysts, emulsifiers, etc., if present) is more than 10%, 20%, 30%, 40%, or 50%, based on the total weight of the reaction mixture.

The term “under an anhydrous condition”, as used herein with respect to a reaction, means the reaction is conducted in the substantial absence of water. In some embodiments, it means the reaction is conducted in the absence of an aqueous phase. In some embodiments, it means the water content in the reaction mixture (including reactants, products, solvents, catalysts, emulsifiers, etc., if present) is no more than 10%, 5%, 2%, 1%, 0.5%, 0.2%, 0.1%, 0.05%, 0.02%, or 0.01%, based on the total weight of the reaction mixture.

The term “anhydrous” as used herein with respect to a material, means the material is substantially free of or free of water. In some embodiments, it means the water content in the material is no more than 10%, 5%, 2%, 1%, 0.5%, 0.2%, 0.1%, 0.05%, 0.02%, or 0.01%, based on the weight of the material.

The term “sustainable” as used herein with respect to a material, such as microcapsule shell and/or active material (e.g., fragrance ingredients), means “biobased” material, which are atoms or molecules obtained from biomass, e.g., obtained from materials containing organic carbon of renewable origin. Source of such carbon can be derived from agricultural products, plants, animals, fungi, microorganisms, marine or forestry materials.

### Polyisocyanate

As used herein, the terms “polyfunctional isocyanate” and “polyisocyanate” can be used interchangeably and refer to a compound having two or more isocyanate (—NCO) groups. Polyisocyanates can be aromatic, aliphatic, linear, branched, or cyclic. In some embodiments, the polyisocyanate contains, on average, 2 to 4 isocyanate groups. In some embodiments, the polyisocyanate contains at least three isocyanate functional groups. In some embodiments, the polyisocyanate is water insoluble. In certain aspects, the polyisocyanate is an oligomeric polyisocyanate obtained from hexamethylene diisocyanate (HDI), which is a monomeric diisocyanate. In certain aspects, the polyisocyanate is an oligomeric polyisocyanate having a biuret, isocyanurate, allophanate, uretdione and/or oligomeric HDI structure. Exemplary polyisocyanates are sold under the tradenames TAKENATE® (e.g., TAKENATE® D-110N; Mitsui Chemicals), DESMODUR® (Covestro), BAYHYDUR® (Covestro), and LUPRANATE® (BASF).

In some embodiments, the polyisocyanate is an aromatic polyisocyanate. Desirably, the aromatic polyisocyanate includes a phenyl, tolyl, xylyl, naphthyl or diphenyl moiety as the aromatic component. In some embodiments, the aromatic polyisocyanate is selected from the group consisting of polyisocyanurate of toluene diisocyanate, trimethylol propane-adduct of toluene diisocyanate, trimethylol propane-adduct of xylylene diisocyanate, and mixtures thereof.

In some embodiments, the aromatic polyisocyanate has the structural formula shown below, and includes structural isomers thereof

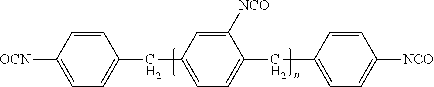

wherein n can vary from zero to a desired number (e.g., 0-50, 0-20, 0-10, or 0-6). Preferably, the number of n is limited to less than 6. The polyisocyanate may also be a mixture of polyisocyanates where the value of n can vary from 0 to 6. In the case where the polyisocyanate is a mixture of various polyisocyanates, the average value of n preferably falls in between 0.5 and 1.5.

In some embodiments, the aromatic polyisocyanate has the structural formula shown below, and includes structural isomers thereof

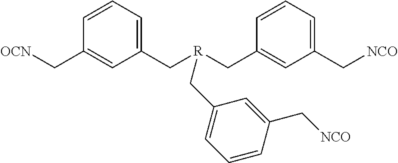

wherein R can be a C₁-C₁₀ alkyl, C₁-C₁₀ ester, or an isocyanurate. Representative polyisocyanates having this structure are sold under the trademarks TAKENATE® D-110N (Mitsui), DESMODUR® L75 (Covestro), and DESMODUR® IL (Covestro).

In some embodiments, the aromatic polyisocyanate is selected from the group consisting of 1,5-naphthylene diisocyanate, 4,4′-diphenylmethane diisocyanate (MDI), hydrogenated MDI (H12MDI), xylylene diisocyanate (XDI), tetramethylxylol diisocyanate (TMXDI), 4,4′-diphenyldimethylmethane diisocyanate, di- and tetraalkyldiphenylmethane diisocyanate, 4,4′-dibenzyl diisocyanate, 1,3-phenylene diisocyanate, 1,4-phenylene diisocyanate, the isomers of tolylene diisocyanate (TDI), 4,4′-diisocyanatophenylperfluoroethane, phthalic acid bisisocyanatoethyl ester, aromatic polyisocyanates with reactive halogen atoms, and mixtures thereof. In some embodiments, the aromatic polyisocyanate with reactive halogen atom is selected from the group consisting of 1-chloromethylphenyl 2,4-diisocyanate, 1-bromomethyl-phenyl 2,6-diisocyanate, 3,3-bischloromethyl ether 4,4′-diphenyldiisocyanate, and mixtures thereof.

In some embodiments, the polyisocyanate is an aliphatic polyisocyanate. In some embodiments, the aliphatic polyisocyanate is selected from the group consisting of trimer of hexamethylene diisocyanate, trimer of isophorone diisocyanate, biuret of hexamethylene diisocyanate, and mixtures thereof. In some embodiments, the aliphatic polyisocyanate is selected from the group consisting of 1-methyl-2,4-diisocyanatocyclohexane, 1,6-diisocyanato-2,2,4-trimethylhexane, 1,6-diisocyanato-2,4,4-trimethylhexane, 1-isocyanatomethyl-3-isocyanato-1,5,5-trimethylcyclohexane, chlorinated aliphatic diisocyanates, brominated aliphatic diisocyanates, phosphorus-containing aliphatic diisocyanates, tetramethoxybutane 1,4-diisocyanate, butane 1,4-diisocyanate, hexane 1,6-diisocyanate (HDI), dicyclohexylmethane diisocyanate, cyclohexane 1,4-diisocyanate, ethylene diisocyanate, and mixtures thereof. In some embodiments, the polyisocyanate comprises a sulfur-containing polyisocyanate which can be obtained, for example, by reacting hexamethylene diisocyanate with thiodiglycol or dihydroxydihexyl sulfide. In some embodiments, the polyisocyanate is an aliphatic diisocyanate selected from the group consisting of trimethylhexamethylene diisocyanate, 1,4-diisocyanatobutane, 1,2-diisocyanatododecane, dimer fatty acid diisocyanate, and mixtures thereof.

In some embodiments, the weight average molecular weight of the polyisocyanate ranges from 250 Da to 1000 Da, or from 275 Da to 500 Da. In some embodiments, the polyisocyanate used in the preparation of the isocyanate-functionalized prepolymer is a single polyisocyanate. In other embodiments, the polyisocyanate is a mixture of polyisocyanates. In some embodiments, the mixture of polyisocyanates includes an aliphatic polyisocyanate and an aromatic polyisocyanate.

### Amphiphilic Compound

An amphiphilic compound is a chemical compound having both hydrophilic and lipophilic groups. In some embodiments, the amphiphilic compound contains at least two functional groups selected from the group consisting of amine groups, hydroxyl groups, carboxyl groups, and combinations thereof. For example, an amphiphilic compound can contain three hydroxyl groups or one amine group and one hydroxyl group. In some embodiments, the amphiphilic compound is selected from the group consisting of partially neutralized acid esters, polyvinyl alcohol, glycolipids, fatty acids, saponins, quillaia extract, surfactant salts having carboxylate and/or linear alcohol groups, and mixtures thereof. In some embodiments, the partially neutralized acid ester is a partially neutralized citric acid ester.

### Biopolymer

The term “biopolymer”, as used herein, means a polymer obtained from a natural source (e.g., plant, fungus, bacterium or animal) or modified biopolymer thereof. The biopolymer can be a polypeptide (e.g., protein) or a polysaccharide. In some embodiments, the biopolymer is soluble or dispersible in an oil phase. In some embodiments, the biopolymer is a polyelectrolyte. A biopolymer can contain amine, hydroxyl and/or carboxyl functional groups which can react with a polyisocyanate.

In some embodiments, the biopolymer is selected from the group consisting of gelatin, collagen, chitosan, modified guar, modified glucan, gum Arabic (gum acacia), modified gum Arabic (modified gum acacia), proteins (e.g., pea protein), hydrolyzed proteins (e.g., hydrolyzed pea protein), fermented proteins, hydrophobin, enzymes, alginate, carrageenan, pectin, modified starch, modified cellulose, and mixtures thereof. In some embodiments, the biopolymer is gelatin. In some embodiments, the biopolymer is chitosan.

As used herein, the term “chitosan” means a linear polysaccharide composed of randomly distributed β-(1-4)-linked D-glucosamine (deacetylated unit) and N-acetyl-D-glucosamine (acetylated unit). Important characteristics in determining the functionality of chitosan are the degree of deacetylation (DDA) and weight average molecular weight (M_{w}). Chitosan is technically defined as chitin with more than 60% DDA. Chitosan with higher percentage DDA possesses more positively charged amine groups when dissolved in solution.

The chitosan is derived from shellfish chitosan or fungal chitosan. In some embodiments, the chitosan is a fungal chitosan. In traditionally sourced crustacean-based chitosan production, large volumes of high temperature, caustic solution are required to chemically remove the acetyl groups. Fungal production of chitosan allows for very high percentage DDA values due to the nature of the fermentation, with the ability to routinely produce a fungal chitosan with DDA as high as 99%. In preferred aspects, chitosan is obtained from a fungal source or derived from fungal chitin by chemical deacetylation. Exemplary fungal sources that may be used in the preparation of fungal chitosan include, but are not limited to, Pleurotus ostreatus and Aspergillus niger.

In certain aspects, the chitosan has a degree of deacetylation (DDA) of from 50% to 95%, preferably from 65% to 90%. In other aspects, the chitosan has a DDA of at least 80%. In certain aspects, the chitosan has weight average molecular weight of from 500 Da to 1,000,000 Da, preferably from 2,000 Da to 500,000 Da, more preferably from 10,000 Da to 400,000 Da, or most preferably from 50,000 Da to 250,000 Da.

### Active Material

The core of the biodegradable core-shell microcapsule comprises at least one active material, which may also serve as the medium for carrying out the isocyanate-functionalized prepolymer formation. In some embodiments, the active material is hydrophobic. In some embodiments, the active material has a log P value (partition coefficient) of less than 2. The active material of the present disclosure includes, but are not limited to, fragrances, flavors, agricultural actives, pesticides, insecticides, herbicides, fungicides, pharmaceutical actives, nutraceutical actives, animal nutrition actives, food actives, microbio actives, malodor counteractants, and/or cosmetic actives.

The present disclosure is also based, inter alia, on the discovery of High Performance fragrance ingredients composed of certain Ultra High-Impact fragrance ingredients and High-Impact fragrance ingredients to deliver improved perceived intensity, perceived longevity and/or perceived fidelity of the fragrance profile at the various “touch points” (e.g., opening a fabric conditioner container, damp clothes upon opening a washing machine after washing laundry, opening a laundry dryer after drying laundry, drying clothes on drying frame and wearing laundered clothes) associated with the laundry experience. In some embodiments, the active material comprises a fragrance, preferably a fragrance comprising at least one, at least two, at least three, at least four, at least five, at least six, at least seven, at least eight, at least nine or at least ten High Performance fragrance ingredients selected from the group consisting of Ultra High-Impact fragrance ingredients as listed in Table 1 and High-Impact fragrance ingredients as listed in Table 2.

TABLE 1
Ultra High-Impact Fragrance Ingredients

| No. | Trade Name | CAS |
|---|---|---|
| 1. | ALD C-11 INTRELEVEN (TT) PRG | 143-14-6 |
| 2. | ALD C-6 TOCO | 66-25-1 |
| 3. | Musk Indenofuran (2,2,6,6,7,8,8- | 476332-65-7, |
| | heptamethyldecahydro-2H-indeno[4,5-b]furan) | 647828-16-8 |
| 4. | AMBERMOR EX | 6790-58-5 |
| 5. | AMBERMORKETAL | 57345-19-4 |
| 6. | AMBROCENIDE CRYST | 211299-54-6 |
| 7. | CALONE | 28940-11-6 |
| 8. | CITRYLAL BHT | 90480-35-6, |
| | | 147060-73-9 |
| 9. | CLONAL | 2437-25-4 |
| 10. | CREOSOL #340 | 93-51-6 |
| 11. | CRESYL ACET PARA COEUR | 140-39-6 |
| 12. | DECENAL, CIS-4 | 21662-09-9 |
| 13. | DODECENAL TRANS-2 TOCO | 20407-84-5 |
| 14. | DUPICAL TOCO | 30168-23-1 |
| 15. | DYNASCONE BHT | 56973-85-4 |
| 16. | FLORAL SUPER | 71077-31-1 |
| 17. | FRAGARONE | 16957-70-3 |
| 18. | GALBASCONE ALPHA 95 PRG | 56973-85-4 |
| 19. | GALBASCONE PRG TOCO | 56973-85-4, |
| | | 56973-84-3 |
| 20. | ISO BUTYL QUINOLINE | 67634-06-4 |
| 21. | ISO BUTYL THIAZOLE | 18640-74-9 |
| 22. | JAMUNATE | 7605-52-9 |
| 23. | KOUMALACTONE 10 PCT TEC FIRM | 77-93-0 |
| 24. | MANGONE CSM | 38462-22-5 |
| 25. | MELOZONE | 30772-79-3 |
| 26. | METH HEPTIN CARBONATE | 111-12-6 |
| 27. | METH LAITONE 10 PCT DPG | 110-98-5 |
| 28. | METH PHEN ETH ETHER | 3558-60-9 |
| 29. | NONADIENAL, 2-TR-6-CIS TOCO | 557-48-2 |
| 30. | NONENOL, CIS-6 TOCO | 35854-86-5 |
| 31. | OPALENE TOCO | 174155-47-6 |
| 32. | ORENYLE | 29127-83-1 |
| 33. | ORRIS ALD TOCO | 60784-31-8 |
| 34. | OXANE 50 PCT TEC | 59323-76-1, |
| | | 59324-17-3 |
| 35. | PASSION FRUIT CMPD | 59323-76-1 |
| 36. | PHARAONE 10 PCT DPG | 110-98-5 |
| 37. | PYRAZINE 044 (METH) | 109-08-0 |
| 38. | ROSE OXIDE “L” TOCO | 16409-43-1 |
| 39. | ROSYRANE SUPER | 60335-71-9 |
| 40. | SACRAZOLE-018 | 137-00-8 |
| 41. | THIAZOLE (2-ISO PROP 4-METH) | 15679-13-7 |
| 42. | TRIDECENE-2-NITRILE | 22629-49-8 |
| 43. | TROPICALIA TOCO | 10138-32-6 |
| 44. | VIONIL NEAT | 67019-89-0 |
| 45. | HEALINGWOOD ¹ | Natural Oil |
| 46. | VIOLET LEAF ABS EGYPT LMR ¹ | Natural Oil |

¹ Available from International Flavors & Fragrances Inc.

TABLE 2
High-Impact Fragrance Ingredients

| No. | Trade Name | CAS |
|---|---|---|
| 1. | ACETOPHENONE | 98-86-2 |
| 2. | AGRUNITRILE | 51566-62-2 |
| 3. | ALD AA TRIPLAL BHT | 68039-49-6 |
| 4. | ALD C-10 | 112-31-2 |
| 5. | ALD C-11 MOA BHT | 19009-56-4 |
| 6. | ALD C-11 ULENIC TOCO | 112-45-8 |
| 7. | ALD C-11 UNDECYLIC TOCO | 112-44-7 |
| 8. | ALD C-12 LAURIC TOCO | 112-54-9 |
| 9. | ALD C-12 MNA TOCO | 110-41-8 |
| 10. | ALD C-16 STRAWB#2 | 77-83-8 |
| 11. | ALD C-18 | 104-61-0 |
| 12. | ALD C-8 TOCO | 124-13-0 |
| 13. | ALD C-9 TOCO | 124-19-6 |
| 14. | AMBERTONIC | 1392325-86-8 |
| 15. | AMBRETTOLIDE | 28645-51-4 |
| 16. | AQUAFLORA TOCO | 1339119-15-1 |
| 17. | BENZALD FFC | 100-52-7 |
| 18. | CASHMERAN | 33704-61-9 |
| 19. | CEDRAMBER | 67874-81-1 |
| 20. | CITRAL NEW | 5392-40-5 |
| 21. | COOLWOOD | 1340502-69-3 |
| 22. | CRESOL PARA EXTRA | 106-44-5 |
| 23. | CRISTALFIZZ | 1093653-57-6 |
| 24. | CYCLAPROP | 68912-13-0 |
| 25. | CYCLEMAX | 7775-00-0 |
| 26. | DAMASCENONE TOCO | 23696-85-7 |
| 27. | DAMASCONE DELTA | 71048-82-3 |
| 28. | DELPHONE | 4819-67-4 |
| 29. | DIPHEN OXIDE | 101-84-8 |
| 30. | DORIFFOX | 149713-23-5 |
| 31. | ETH ACETO ACET | 141-97-9 |
| 32. | ETH CAPROATE | 123-66-0 |
| 33. | ETH PHEN GLYC | 121-39-1 |
| 34. | ETH SAL | 118-61-6 |
| 35. | ETH-2-METH BUTY | 7452-79-1 |
| 36. | EUCALYPTOL USP | 470-82-6 |
| 37. | FLORHYDRAL TOCO (ELINCS) | 125109-85-5 |
| 38. | FRUITATE (ELINCS) | 129520-41-8 |
| 39. | HEXADECANOLIDE BHT | 109-29-5 |
| 40. | HEXENYL ISOBUTY, CIS-3 (VERDURAL B) | 41519-23-7 |
| 41. | INDOLE | 120-72-9 |
| 42. | IONONE ALPHA TOCO | 127-41-3 |
| 43. | IRONE V BHT | 79-69-6 |
| 44. | ISO CYCLO CITRAL TOCO | 1423-46-7 |
| 45. | LACTONE OF CIS JASMONE TOCO | 70851-61-5 |
| 46. | LEMONILE | 61792-11-8 |
| 47. | MELONAL TOCO | 106-72-9 |
| 48. | MENTHONE 85 | 89-80-5 |
| 49. | METH BENZOATE | 93-58-3 |
| 50. | METH CINNAMATE TOCO | 103-26-4 |
| 51. | METH DH JASMONATE | 24851-98-7 |
| 52. | METH HEPTYL KETONE | 821-55-6 |
| 53. | METH JASMONATE TOCO | 39924-52-2 |
| 54. | METH OCTIN CARBONATE | 111-80-8 |
| 55. | METH PARA CRESOL | 104-93-8 |
| 56. | METH TUBERATE RD | 35205-76-6 |
| 57. | MUSCEMOR (ELINCS) | 82356-51-2 |
| 58. | MYRAC ALD BHT | 52475-89-5 |
| 59. | NECTARYL LRG 1371 ELINCS | 95962-14-4 |
| 60. | OCEANOL | 33662-58-7 |
| 61. | OPERANIDE (ELINCS) | 823178-41-2 |
| 62. | PHEN ETH ACET | 103-45-7 |
| 63. | PINEAPPLE CMPD | 3658-77-3 |
| 64. | PINO ACETALD TOCO | 33885-51-7 |
| 65. | ROSALVA | 13019-22-2 |
| 66. | ROSE OXIDE TOCO | 16409-43-1 |
| 67. | ROSETHYL | 64988-06-3 |
| 68. | SAFRALEINE | 54440-17-4 |
| 69. | SINFONIDE | 1315251-11-6 |
| 70. | STARFLEUR TOCO | 1254940-85-6 |
| 71. | SYLVONIC | 98-55-5 |
| 72. | TERPINOLENE P UB BHT | 586-62-9 |
| 73. | TOFFEE LACTONE 2067 | 705-86-2, |
| | | 706-14-9 |
| 74. | TOFFEETONE FOR NON TSCA USE ONLY | 13537-82-1 |
| 75. | TONKALACTONE | 54491-17-7 |
| 76. | TRIFERNAL BHT | 16251-77-7 |
| 77. | ULTRAVANIL Q COLIPA | 2563-07-7 |
| 78. | UNDECALACTONE, DELTA | 710-04-3 |
| 79. | UNDECAVERTOL TOCO | 81782-77-6 |
| 80. | VANITROPE | 94-86-0 |
| 81. | VARAMOL-106 | 7786-61-0 |
| 82. | VELTOL PLUS | 4940-11-8 |
| 83. | VERAMOSS | 4707-47-5 |
| 84. | VERIDIAN | 811412-48-3 |
| 85. | VERTOLIFF TOCO | 36635-35-5 |
| 86. | VERTONIC | 1945993-03-2 |
| 87. | VETIVER ACET HAITI TOCO BLO | 84082-84-8 |
| 88. | YARA YARA | 93-04-9 |
| 89. | YLANGANATE | 89-71-4 |
| 90. | ARMOISE OIL PURE ¹ | Natural oil |
| 91. | BLKCURRANT BUD ABS BURGUNDY | Natural oil |
| | LMR FOR LIFE ¹ | |
| 92. | CHAMOMILE OIL ENG ³ | Natural oil |
| 93. | CHAMOMILE OIL ROMAN LMR SFO ¹ | Natural oil |
| 94. | CINNAMON BARK ESSENTIAL LMR ¹ | Natural oil |
| 95. | DAVANA OIL LMR FLG SFO ¹ | Natural oil |
| 96. | EUGENOL NAT EX CLOVE LEAF OIL | Natural oil |
| 97. | JASMIN ABS EGYPT LMR ¹ | Natural oil |
| 98. | ORANGE FLOWER WATER ABS TUNISIA | Natural oil |
| | LMR ¹ | |
| 99. | OSMANTHUS ABS LMR ¹ | Natural oil |
| 100. | PATCHOULI OIL LIGHT BLO ² | Natural oil |
| 101. | PEPPER PINK CO2 LMR ¹ | Natural oil |
| 102. | ROSE ABS DAMASCENA PURE BLO ¹ | Natural oil |
| 103. | SINENSAL NATURAL 20 EX ORANGE ³ | Natural oil |
| 104. | THYME OIL WHITE SPAIN BLO ¹ | Natural oil |

¹ Available from International Flavors & Fragrances Inc (New York).
² Available from TRIPPER PTE Ltd. (Indonesia).
³ Available from TREATT & CO. Ltd. (United Kingdom).

In addition to the fragrance ingredients listed in Tables 1 and 2, the fragrance May further comprise at least one additional fragrance ingredient. Preferably, the fragrance may further comprise 1, 2, 3, 4, 5, 6, 7, 8, 9, 10, 15, 20, 25, 30, 35, 40 or more additional fragrance ingredients, which are not listed in Tables 1 and 2. Non-limiting examples of such additional fragrance ingredients include those described in US 2018/0325786 A1, U.S. Pat. Nos. 4,534,891, 5,112,688, and 5,145,842. Other suitable active materials that can be encapsulated include those listed in WO 2016/049456, pages 38-50.

In some embodiments, the additional fragrance ingredients, when combined with one or more fragrance ingredients of Tables 1 and 2, constitute the total fragrance composition. In this respect, the balance of the 100 wt % relative to the total weight of the fragrance component is made up of one or more Ultra High-Impact and High-Impact fragrance ingredients of Tables 1 and 2 and one or more additional fragrance ingredients.

It has been surprisingly discovered that a fragrance that includes certain types of Ultra High-Impact and High-Impact fragrance ingredients results in High Performance fragrance ingredients for use in fabric care composition, preferably fabric conditioner, that improves fragrance profile and/or performance of the system. As used herein, the term “fragrance profile” means the description of how the fragrance is perceived by the human nose at any moment in time. The fragrance profile may change over time. It is a result of the combination of the base, heart and top notes, if present, of a fragrance. Base notes are characterized by providing animalic, woody, sweet, amber or musky characters, and not being very volatile. Heart notes are associated with desirable characters such as floral characters (e.g., jasmine, rose), fruity, marine, aromatic or spicy characters. The “top or head notes” provide citrusy, green, light, or fresh characters and tend to evaporate quickly due to their high volatility. A fragrance profile is composed of 2 characteristics: “intensity” and “character”. The “intensity” relates to the perceived strength whilst “character” refers to the odor impression or quality of the perfume, i.e., fresh, clean, etc.

In some embodiments, the fragrance of the present disclosure can be used at a dosage level of ≤1 wt % relative to the total weight of the consumer product composition without significantly impacting the fragrance profile, i.e., perceived fragrance intensity, perceived fragrance longevity and/or perceived fragrance fidelity, particularly for select characters (e.g., fresh and/or clean). In some aspects, the fragrance is used at a dosage level of less than or equal to 1 wt %, 0.99 wt %, 0.95 wt %, 0.9 wt %, 0.8 wt %, 0.7 wt %, 0.6 wt %, 0.5 wt %, 0.4 wt %, 0.3 wt %, 0.2 wt %, 0.1 wt %, 0.05 wt % or 0.01 wt % of the total weight of the aqueous fabric conditioner composition, or any range delimited by any pair of the foregoing values.

In some embodiments, the active material comprises agricultural active, pesticide, insecticide, herbicide, and/or fungicide. Encapsulation of agricultural actives, pesticides, insecticides, herbicides, and/or fungicides can increase their efficacy, extend their effective period and protect the environment. In some embodiments, the insecticide is an organophosphate insecticide. In some embodiments, the insecticide can be an organophosphate insecticide selected from the group consisting of acephate, azinphos-mehyl, chlorfenvinphos, chlorethoxyfos, chlorpyriphos-methyl, diazinon, dimethoate, disulfoton, ethoprophos, fenitrothion, fenthiom, fenamiphos, fosthiazate, malathion, methamidophos, methidathion, omethoate, oxydemeton-methyl, parathion, parathion-methyl, phorate, phosmet, profenofos, trichlorfon, and mixtures thereof. In some embodiments, the insecticide is selected from the group consisting of cypermethrin, bifenthrin, λ-cyhalothrin, and mixtures thereof. In some embodiments, the herbicide is selected from the group consisting of clomazone, acetochlor, pendimethalin, and mixtures thereof. In some embodiments, the fungicide is tebuconazole.

### Polyelectrolyte

The polyelectrolyte of the present disclosure is a biopolymer. In some embodiments, the polyelectrolyte is selected from the group consisting of gelatin, collagen, chitosan, modified guar, modified glucan, gum Arabic (gum acacia), modified gum Arabic (modified gum acacia), proteins (e.g., pea protein), hydrolyzed proteins (e.g., hydrolyzed pea protein), fermented proteins, hydrophobin, enzymes, partially neutralized citric acid ester, alginate, carrageenan, pectin, modified starch, modified cellulose, and mixtures thereof. In some embodiments, the polyelectrolyte comprises gum Arabic. In some embodiments, the polyelectrolyte comprises alginate. The polyelectrolyte has a biodegradation rate of at least 20%, 30%, 40%, 50%, 60%, 65%, 70%, 75%, 80%, 85%, 90%, 95% or 98%, based on the weight of the polyelectrolyte, within 60 days according to OECD301F.

### First Crosslinker

Crosslinker is a crosslinking agent. In some embodiments, the first crosslinker comprises an oxidized sugar and/or an enzyme, wherein the oxidized sugar comprises aldehyde groups, and the enzyme is selected from the group consisting of transglutaminase, laccase, peroxidase, oxidase, amylase, transferase, and mixtures thereof. In some embodiments, the first crosslinker comprises an enzyme selected from the group consisting of transglutaminase, laccase, peroxidase, oxidase, amylase, transferase, and mixtures thereof. In some embodiments, the first crosslinker comprises an oxidized sugar and an enzyme. In some embodiments, the enzyme is transglutaminase. In some embodiments, the first crosslinker comprises transglutaminase.

In some embodiments, the first crosslinker comprises an oxidized sugar having two or more aldehyde groups. Use of an oxidized sugar as a crosslinker has been found to highly crosslink the prepolymer and/or the polyelectrolyte. The term “oxidized sugar”, as used herein, means a sugar (e.g., monosaccharide, oligosaccharide, and/or polysaccharide) that has been treated with an oxidizing agent to generate reactive aldehyde groups. In some embodiments, the sugar has a molecular weight of less than 1000 g/mol and is soluble or dispersible in an aqueous phase. In some embodiments, the sugar is selected from the group consisting of glucose, glucosamine, sucrose, maltose, lactose, maltodextrin, cyclodextrin, polysaccharide, hydrolyzed polysaccharide, and mixtures thereof. In some embodiments, the sugar is selected from the group consisting of sucrose, glucosamine, maltodextrin, cyclodextrin, and mixtures thereof. In some embodiments, the sugar comprises sucrose. In some embodiments, the oxidizing agent is selected from the group consisting of sodium periodate, hydrogen peroxide, laccases, oxidases, and combinations thereof. In some embodiments, the oxidizing agent comprises sodium periodate.

### Second Crosslinker

In some embodiments, the second crosslinker is selected from the group consisting of tannic acid, hydrolyzed tannic acid, tannin, gallic acid, methyl gallate, ethyl gallate, glutaraldehyde, glyoxal, triethyl citrate, malondialdehyde, genipin, dopamine, phenols, polyphenols, polycarbodiimide, polyacid chlorides, tetraethoxysilane, enzymes, multivalent cations, and combinations thereof. The tannic acid includes hydrolyzed and unhydrolyzed tannic acid. In some embodiments, the second crosslinker comprises a tannic acid. In some embodiments, the second crosslinker comprises a glutaraldehyde. Multivalent cations include divalent cations such as Ca2⁺ and Mg²⁺. In some embodiments, the second crosslinker comprises a multivalent cation such as a calcium ion (Ca2⁺). In some embodiments, the enzymes are selected from the group consisting of transglutaminase, laccase, peroxidase, oxidase, amylase, transferase, and mixtures thereof. It has been unexpectedly discovered that the use of a second crosslinker maintains the coacervation properties of the microcapsule in various aqueous solution environments, provides pH stability and temperature stability (e.g, microcapsule is stable at high temperature), and improves performance stability of the microcapsule in a fabric softener.

In some embodiments, the second crosslinker comprises an aldehyde crosslinker having one or more, preferably two or more, formyl groups (—CHO). In some embodiments, the aldehyde crosslinker is selected from the group consisting of glutaraldehyde, glyoxal, genipin (e.g., polymerized genipin), di-aldehyde starch, malondialdehyde, succinic dialdehyde, 1,3-propane dialdehyde, 1,4-butane dialdehyde, 1,5-pentane dialdehyde, 1,6-hexane dialdehyde, glyoxal trimer, paraformaldehyde, bis(dimethyl) acetal, bis(diethyl) acetal, polymeric dialdehydes, and mixtures thereof. In some embodiments, the aldehyde crosslinker is selected from the group consisting of glutaraldehyde, glyoxal, genipin, and mixtures thereof.

In some embodiments, the second crosslinker comprises a phenolic crosslinker having at least two hydroxyphenyl groups. In some embodiments, the phenolic crosslinker is selected from the group consisting of flavonoid, isoflavonoid, neoflavonoid, gallotannin, ellagotannin, catechol, DL-3,4-dihydroxyphenylalaline, catecholamine, dopamine, phloroglucinol, phenolic acid, phenolic ester, phenolic heteroside, curcumin, polyhydroxylated coumarin, polyhydroxylated lignan, neolignan, poly-resorcinol, tannin, hydrolyzed tannic acid, and mixtures thereof. Examples of suitable phenolic acid include gallic acid and tannic acid. Examples of suitable phenolic ester include methyl gallate and ethyl gallate.

In some embodiments, the second crosslinker comprises a polyphenol crosslinker which is a phenolic acid having 3,4,5-trihydroxyphenyl group or 3,4-dihydroxyphenyl group. In some embodiments, the polyphenol crosslinker is selected from the group consisting of tannic acid, hydrolyzed tannic acid, gallic acid, methyl gallate, ethyl gallate, and mixtures thereof.

Conventionally, polyurea-based microcapsules are prepared by first dissolving a polyisocyanate in an oil phase and then emulsifying the oil phase with an aqueous phase containing polyamines, polyelectrolytes or biopolymers thereby promoting the reaction of the polymers at the oil-water interface. While the intended reaction is an interfacial polymerization, controlling the kinetics to yield a copolymer/composite is difficult. It has been observed that capsules prepared via this traditional method yield a blend of polyurea formed from the self-condensation of polyisocyanate coated with a layer of unreacted material originally intended for the interfacial polymerization. The issue with such capsules is that the shell, even though it may pass the OECD301F or OECD310 tests, may still not be “truly” biodegradable, for example under proposed ECHA regulations, because it is considered a blend of biodegradable (i.e., biopolymers) and non-biodegradable materials (i.e., self-condensed polyisocyanate) and therefore the blend would not meet the biodegradability requirements.

It has now been found that when a biopolymer and/or an amphiphilic compound is reacted with a polyisocyanate under an anhydrous condition, an isocyanate-functionalized prepolymer can be formed, which inhibits the self-polymerization of polyisocyanate. Accordingly, the present disclosure provides an isocyanate-functionalized prepolymer which comprises the reaction product of a polyisocyanate with a biopolymer and/or an amphiphilic compound under an anhydrous condition. In some embodiments, the isocyanate-functionalized prepolymer comprises the reaction product of a polyisocyanate with a biopolymer under an anhydrous condition. In some embodiments, the isocyanate-functionalized prepolymer comprises the reaction product of a polyisocyanate with an amphiphilic compound under an anhydrous condition. The present disclosure also provides a process for making the isocyanate-functionalized prepolymer. The present disclosure also provides the isocyanate-functionalized prepolymer obtainable by the process. The process comprises reacting a polyisocyanate with a biopolymer and/or an amphiphilic compound under an anhydrous condition to form the isocyanate-functionalized prepolymer. In some embodiments, the process comprises reacting a polyisocyanate with a biopolymer under an anhydrous condition to form the isocyanate-functionalized prepolymer. In some embodiments, the reaction is conducted in the presence of a catalyst. In some embodiments, the polyisocyanate is dissolved in a solution comprising a solvent and/or an active material. The formed isocyanate-functionalized prepolymer can be used to make the biodegradable core-shell microcapsule of this disclosure.

The term “prepolymer”, as used herein, means a biopolymer and/or an amphiphilic compound that has been reacted to an intermediate state for further reaction (e.g., with a polyelectrolyte) and/or crosslinking (e.g., with a first crosslinker and optionally a second crosslinker). An “isocyanate-functionalized prepolymer” is a prepolymer in which all or a portion of the amine, hydroxyl and/or carboxyl functional groups of a biopolymer and/or an amphiphilic compound have been reacted with a portion of the isocyanate groups of a polyisocyanate rendering isocyanate functionality to the biopolymer and/or the amphiphilic compound. The isocyanate-functionalized prepolymer comprises a biopolymer and/or an amphiphilic compound treated or reacted with a polyisocyanate under an anhydrous condition. In some embodiments, the isocyanate-functionalized prepolymer comprises a biopolymer treated or reacted with a polyisocyanate under an anhydrous condition.

Under an aqueous condition, the isocyanate functional group can react with water to form —NH₂ (via carbamic acid), which can then react with unreacted isocyanate functional group to form urea bond. To inhibit this self-polymerization and formation of self-condensed polyisocyanate and promote prepolymer formation, the polyisocyanate is combined or mixed with the biopolymer and/or the amphiphilic compound under an anhydrous condition (e.g., in oil phase or an anhydrous solution). In some embodiments, the polyisocyanate is dissolved in an anhydrous solution and the biopolymer and/or the amphiphilic compound is likewise dissolved or dispersed therein to form the reaction mixture. The anhydrous solution also comprises an anhydrous solvent, an anhydrous active material, or a combination thereof (i.e., a solvent and an active material). Exemplary solvents or dispersants include, but are not limited to, mineral oil, dimethylsulfoxide (DMSO), dimethylformamide (DMF), benzyl benzoate, triacetin, ethyl acetate, ethylene glycol diacetate, propylene glycol diacetate, diethyl malonate, triethyl citrate, ethyl acetoacetate, benzyl acetone, butyl carbitol acetate, 3-methyl butyl acetate, diethylene glycol monoethyl ether acetate (DGMEA), Aromatic 100 (high solvency C9 aromatic fluid), Aromatic 200 (C10-C13 aromatic fluid), isopropyl myristate, isopropyl palmitate, dioctyl adipate, verdox, iso E super, Hercolyn, amyl benzoate, hexyle benzoate, phenyl ethyl benzoate, cp formate aphermate, caprylic/capric triglyceride, 3-methoxybutyl acetate, and combinations thereof. In some embodiments, the solvent is hydrophobic. In some embodiments, the solvent comprises caprylic/capric triglyceride. In some embodiments, the solvent comprises benzyl benzoate. In some embodiments, the solvent comprises 3-methoxybutyl acetate. In some embodiments, the solvent comprises phenyl ethyl benzoate. The active material used herein in the preparation of the prepolymer is same as the active material contained in the microcapsule core.

In the anhydrous solution, the polyisocyanate is combined or mixed with the biopolymer and/or the amphiphilic compound to form the reaction mixture. The reaction mixture optionally comprises a catalyst. As the reaction proceeds, optionally in the presence of the catalyst, the polyisocyanate reacts with the biopolymer and/or the amphiphilic compound to form an isocyanate-functionalized prepolymer. At the end of the reaction, the reaction mixture becomes a product mixture comprising the isocyanate-functionalized prepolymer. The product mixture also comprises the solvent, the active material, or a combination thereof. In some embodiments, the product mixture comprises both the solvent and the active material. In some embodiments, the reaction is conducted at a pH of from 1 to 12, preferably from 3 to 5.5. In some embodiments, the reaction temperature is from 25° C. to 250° C., preferably from 40° C. to 60° C. In some embodiments, at the end of the reaction, the amount of the free polyisocyanate (i.e., unreacted polyisocyanate) in the product mixture is less than 12%, less than 10%, less than 5%, less than 3%, less than 1%, less than 0.5%, less than 0.1%, less than 0.05%, or less than 0.01% relative to the total weight of the polyisocyanate used for the reaction. In some embodiments, the amphiphilic compound is not present in the reaction mixture, and the reaction only involves the polyisocyanate with the biopolymer.

Preferably, the prepolymer formation is further facilitated by the inclusion of a catalyst in the reaction. In some embodiments, the catalyst is soluble or dispersible in the anhydrous solution comprising (i) the polyisocyanate and (ii) the biopolymer and/or the amphiphilic compound. The suitable catalyst is capable of catalyzing the formation of urea, urethane, and/or amide reaction products. In some embodiments, the concentration of catalyst is between 0.001% and 1% by weight of the reaction mixture, preferably between 0.01% and 0.02% by weight of the reaction mixture. Preferably, the catalyst contains at least one tertiary amine. Examples of suitable catalysts used in the preparation of the prepolymer include, but are not limited to, 1,4-diazabicylo[2,2,2]octane (DABCO), N-methylimidazole, diaminobicycloctane, 2,2′-dimorpholinodiethylether, or any combination thereof. Preferably, the catalyst comprises DABCO.

Formation of the isocyanate-functionalized prepolymer is evident by an increase in the molecular weight of the biopolymer and/or the amphiphilic compound. The final molecular weight of the prepolymer may vary depending on the kind of reactants, the amount/ratio of reactants, and/or the reaction condition. The formed isocyanate-functionalized prepolymer has a biodegradation rate of at least 20%, 30%, 40%, 50%, 60%, 65%, 70%, 75%, 80%, 85%, 90%, 95% or 98%, based on the weight of the isocyanate-functionalized prepolymer, within 60 days according to OECD301F.

The present disclosure also provides a biodegradable core-shell microcapsule comprising: (a) a microcapsule shell comprising the reaction product of an isocyanate-functionalized prepolymer with a crosslinker and optionally a polyelectrolyte under an aqueous condition; and (b) a microcapsule core comprising an active material; wherein the isocyanate-functionalized prepolymer is the reaction product of a polyisocyanate with a biopolymer and/or an amphiphilic compound under an anhydrous condition, the biopolymer is selected from the group consisting of gelatin, collagen, chitosan, modified guar, modified glucan, gum Arabic (gum acacia), modified gum Arabic (modified gum acacia), proteins (e.g., pea protein), hydrolyzed proteins (e.g., hydrolyzed pea protein), fermented proteins, hydrophobin, enzymes, alginate, carrageenan, pectin, modified starch, modified cellulose, and mixtures thereof, the amphiphilic compound is selected from the group consisting of partially neutralized acid esters, polyvinyl alcohol, glycolipids, fatty acids, saponins, quillaia extract, surfactant salts having carboxylate and/or linear alcohol groups, and mixtures thereof, the microcapsule shell has a biodegradation rate of at least 20%, 30%, 40%, 50%, 60%, 65%, 70%, 75%, 80%, 85%, 90%, 95% or 98%, based on the weight of the microcapsule shell, within 60 days according to OECD301F, and the microcapsule shell is substantially free of or free of a self-condensed polyisocyanate. In some embodiments, the isocyanate-functionalized prepolymer is the reaction product of a polyisocyanate with a biopolymer under an anhydrous condition. In some embodiments, the isocyanate-functionalized prepolymer is the reaction product of a polyisocyanate with an amphiphilic compound under an anhydrous condition. In some embodiments, the partially neutralized acid ester is a partially neutralized citric acid ester.

The biodegradable core-shell microcapsule of the present disclosure has a core comprising an active material, and a shell comprising an isocyanate-functionalized prepolymer (preferably crosslinked) and optionally a polyelectrolyte (preferably crosslinked). The term “biodegradable” as used herein with respect to a material, such as a microcapsule shell as a whole or a polymer (e.g., biodegradable polymer or prepolymer) of the microcapsule shell, means that the material has no real or perceived health and/or environmental issues, and is capable of undergoing and/or does undergo physical, chemical, thermal, microbial, biological and/or UV or photo-degradation. Ideally, a microcapsule shell and/or polymer is deemed “biodegradable” when the microcapsule shell and/or polymer passes one or more of the following tests including: a respirometry biodegradation method in aquatic media, available from Organization for Economic Cooperation and Development (OECD), International Organization for Standardization (ISO) and the American Society for Testing and Material (ASTM) tests including, but not limited to OECD 301F or 310 (Ready biodegradation), OECD 302 (inherent biodegradation), ISO 17556 (solid stimulation studies), ISO 14851 (fresh water stimulation studies), ISO 18830 (marine sediment stimulation studies), OECD 307 (soil stimulation studies), OECD 308 (sediment stimulation studies), and OECD 309 (water stimulation studies). Preferably, the microcapsules are readily biodegradable as determined using a respirometry biodegradation method in aquatic media, the OECD 301F or OECD 310 test. More preferably, the shell and/or prepolymer of the microcapsule is biodegradable if the shell and/or prepolymer has a biodegradation rate of at least 20%, 30%, 40%, 50%, 60%, 65%, 70%, 75%, 80%, 85%, 90%, 95% or 98%, based on the weight of the shell and/or prepolymer, within 60 days according to the OECD301F or OECD310 tests, or most preferably a biodegradability of at least 60% within 60 days according to OECD301F test.

As used herein, the terms “capsule”, “microcapsule” and “core-shell microcapsule” are used interchangeably and refer to a substantially spherical structure having a well-defined core and a well-defined envelope or wall or shell. The “core” comprises an active material or material submitted to microencapsulation. The terms “wall” and “shell” are used interchangeably to denote the structure formed by the microencapsulating polymer surrounding the active material core being microencapsulated. In general, the wall of the microcapsule is made of a continuous, polymeric phase with an inner surface and outer surface. The inner surface is in contact with the microcapsule core. The outer surface is in contact with the environment in which the microcapsule resides, e.g., a water phase, skin, or hair. Ideally, the wall protects the core against deterioration by oxygen, moisture, light, and effect of other compounds or other factors; limits the losses of volatile core materials; and releases the core material under desired conditions. In this respect, the core-shell microcapsule of the present disclosure provides controlled release and/or diffusional release of the active material. As used herein, “controlled release” refers to retention of the active material in the core until a specified triggering condition occurs. Such triggers include, e.g., friction, swelling, a pH change, an enzyme, a change in temperature, a change in ionic strength, or a combination thereof.

The core of the biodegradable core-shell microcapsule comprises at least one active material. In some embodiments, the microcapsule comprises at least two, three, four or more active materials in the core. In some embodiments, the active material is anhydrous. In some embodiments, the active material is soluble or dispersible in a solvent. The active material is encapsulated in the core-shell microcapsule. In some embodiments, the active material component (e.g., fragrance) of the core-shell microcapsule is present at between 5.0% to 90.0%, preferably between 10.0% to 40.0%, by weight of the microcapsule. When the fragrance comprises a combination of the Ultra High-Impact fragrance ingredients and the High-Impact fragrance ingredients listed in Tables 1 and 2, the fragrance can be used in an aqueous fabric conditioner product at a significantly reduced dosage (e.g., at least 2-, 5- to 10-fold lower levels) as compared to a standard fragrance that does not include the Ultra High-Impact fragrance ingredients and High-Impact fragrance ingredients listed in Tables 1 and 2.

In addition to the active materials, the present disclosure also contemplates the incorporation of additional components including core modifier materials in the core encapsulated by the microcapsule wall. Other components include solubility modifiers, density modifiers, stabilizers, viscosity modifiers, pH modifiers, deposition aids, capsule formation aids, catalysts, processing aids or any combination thereof. These components can be present in the wall or core of the microcapsule, or outside the microcapsule in a microcapsule slurry to improve solubility, stability, deposition, capsule formation, and the like. Further, the additional components may be added after and/or during the preparation of the microcapsule slurry of the present disclosure.

The shell of the biodegradable core-shell microcapsule comprises the reaction product of an isocyanate-functionalized prepolymer with a crosslinker and optionally a polyelectrolyte under an aqueous condition. The crosslinker comprises a first crosslinker and optionally a second crosslinker. In some embodiments, the microcapsule shell comprises an isocyanate-functionalized prepolymer crosslinked with a first crosslinker and optionally a second crosslinker. In some embodiments, the microcapsule shell comprises an isocyanate-functionalized prepolymer and a polyelectrolyte. In some embodiments, the isocyanate-functionalized prepolymer and the polyelectrolyte form a coacervate. In some embodiments, the coacervate is crosslinked with a first crosslinker and optionally a second crosslinker. In some embodiments, the isocyanate-functionalized prepolymer and the polyelectrolyte are covalently bonded or crosslinked. In some embodiments, the isocyanate-functionalized prepolymer and the polyelectrolyte are covalently bonded or crosslinked via the polyisocyanate and the first crosslinker. In some embodiments, the isocyanate-functionalized prepolymer is crosslinked with the first crosslinker and/or the second crosslinker. In some embodiments, the polyelectrolyte is crosslinked with the first crosslinker and/or the second crosslinker. In some embodiments, the isocyanate-functionalized prepolymer is crosslinked with the first crosslinker and optionally the second crosslinker. In some embodiments, the polyelectrolyte is crosslinked with both the first crosslinker and the second crosslinker.

To achieve the desired performance characteristics (e.g., stability, active material retention and controlled release), the microcapsule shell is crosslinked with the crosslinker, that is, the prepolymer and/or the polyelectrolyte is crosslinked with a first crosslinker and optionally a second crosslinker thereby forming the microcapsule wall. In some embodiments, the weight average molecular weight of the crosslinked prepolymer and/or polyelectrolyte is at least 2 times, at least 3 times, at least 4 times, at least 5 times, at least 6 times, at least 7 times, at least 8 times, at least 9 times, or at least 10 times, preferably at least 2 times, at least 4 times, or at least 10 times, of the weight average molecular weight of the non-crosslinked prepolymer and/or polyelectrolyte respectively, as determined by size exclusion chromatography. Advantageously, the resulting microcapsule is pH stable (e.g., pH stable over a pH range of from 1 to 12), high-temperature stable, biodegradable, exhibits a high encapsulation efficiency, and provides high performance in consumer products such as fabric conditioners.

In a microcapsule or a process of making the microcapsule in the present disclosure, the biopolymer and the amphiphilic compound (used to form the isocyanate-functionalized prepolymer) are different from the polyelectrolyte. In some embodiments, the biopolymer comprises gelatin and/or chitosan, and the polyelectrolyte comprises modified guar, gum Arabic (gum acacia), modified gum Arabic (modified gum acacia), alginate, carrageenan, and/or pectin.

In some embodiments, the isocyanate-functionalized prepolymer is the reaction product of a polyisocyanate with a biopolymer under an anhydrous condition, the biopolymer comprises gelatin, the polyelectrolyte comprises gum Arabic, the first crosslinker comprises an oxidized sucrose, and the second crosslinker comprises glutaraldehyde. The gelatin prepolymer is crosslinked with oxidized sucrose and optionally glutaraldehyde, gum Arabic is crosslinked with oxidized sucrose and glutaraldehyde, and the gelatin prepolymer and gum Arabic are covalently bonded or crosslinked via the polyisocyanate and oxidized sucrose.

In some embodiments, the isocyanate-functionalized prepolymer is the reaction product of a polyisocyanate with a biopolymer under an anhydrous condition, the biopolymer comprises gelatin, the polyelectrolyte comprises alginate, the first crosslinker comprises an oxidized sucrose, and the second crosslinker comprises a multivalent cation such as calcium ion. The gelatin prepolymer is crosslinked with oxidized sucrose, alginate is crosslinked with oxidized sucrose and multivalent cation, and the gelatin prepolymer and alginate are covalently bonded or crosslinked via the polyisocyanate and oxidized sucrose.

In some embodiments, the isocyanate-functionalized prepolymer is the reaction product of a polyisocyanate with a biopolymer under an anhydrous condition, the biopolymer comprises chitosan, the polyelectrolyte comprises gum Arabic, the first crosslinker comprises an oxidized sucrose, and the second crosslinker comprises tannic acid. The chitosan prepolymer is crosslinked with oxidized sucrose and optionally tannic acid, gum Arabic is crosslinked with oxidized sucrose and tannic acid, and the chitosan prepolymer and gum Arabic are covalently bonded or crosslinked via the polyisocyanate and oxidized sucrose.

In some embodiments, the core-shell microcapsule comprises between 0.1% and 20%, preferably between 5% and 10%, most preferably 7% by weight of the isocyanate-functionalized prepolymer; between 0.1% and 20%, preferably between 5% and 10%, most preferably 7% by weight of the polyelectrolyte; between 1% and 80%, preferably between 50% and 70%, most preferably 66% by weight of the active material; between 1% and 70%, preferably between 20% and 40%, most preferably 27% by weight of the solvent; between 0.1% and 10%, preferably between 3% and 6%, most preferably 4% by weight of the first crosslinker; and between 0.1% and 5%, preferably between 2% and 3%, most preferably 2% by weight of the second crosslinker; wherein the weights are relative to the total weight of the microcapsule.

In some embodiments, a microcapsule slurry comprising the core-shell microcapsule of the present disclosure is composed of between 0.25% and 10% by weight of the biopolymer, between 0.01% and 2% by weight of the polyisocyanate, between 0.001% and 1% by weight of the catalyst, between 0.1% and 10% by weight of the polyelectrolyte, between 0.01% and 50% by weight of the active material, between 0.01% and 40% by weight of the solvent, between 0.01% and 5% by weight of the first crosslinker, and between 0.01% and 1% by weight of the second crosslinker, wherein the weights are relative to the total weight of the microcapsule slurry.

In some embodiments, one or more non-confined or unencapsulated active materials can also be included in the microcapsule slurry post-curing. Such active materials may be the same or different than the encapsulated active material and may be included at a level of from 0.01% to 20%, preferably from 2% to 10%, based on the total weight of the microcapsule slurry.

In some embodiments, the core-shell microcapsule has a particle size (in diameter) in the range of from 0.1 micron to 1000 microns (e.g., from 0.5 micron to 500 microns, from 1 micron to 200 microns, from 1 micron to 100 microns, or from 1 micron to 50 micron) with a lower limit of 0.1 micron, 0.5 micron, 1 micron, 2 microns, 5 microns or 20 microns, and an upper limit of 1000 microns, 500 microns, 200 microns, 100 microns, 75 microns, 50 microns, 30 microns, 20 microns, 10 microns or 5 microns.

Compared to conventional polyisocyanate and biopolymer microcapsules, which are formed by self-condensed polyisocyanate and a separate layer of biopolymer(s), the wall of microcapsule of the present disclosure is formed from an isocyanate-functionalized prepolymer, thereby substantively or completely eliminating self-condensed polyisocyanate. In some embodiments, the biodegradable core-shell microcapsule of the present disclosure is substantively free of or completely free of self-condensed polyisocyanate and therefore exhibits superior biodegradability properties (i.e., by avoiding and/or reducing blends of biodegradable and non-biodegradable materials). In some embodiments, the biodegradable core-shell microcapsule has a level of self-condensed polyisocyanate that is ≤10%, ≤5%, ≤3%, ≤1%, ≤0.5%, ≤0.1% or ≤0.05%, relative to total weight of polyisocyanate used to form wall of the microcapsules.

In some embodiments, the microcapsule shell is substantially free of or free of a self-condensed polyisocyanate. In some embodiments, the microcapsule shell comprises no more than 10%, 5%, 3%, 1%, 0.5%, 0.1%, or 0.05% self-condensed polyisocyanate, relative to the total weight of polyisocyanate used to form the microcapsule shell. In some embodiments, the microcapsule shell comprises no more than 5%, 3%, 1%, 0.5%, 0.1%, 0.05%, 0.02%, or 0.01% self-condensed polyisocyanate, relative to the total weight of the microcapsule shell. In some embodiments, the core-shell microcapsule comprises no more than 3%, 1%, 0.5%, 0.1%, 0.05%, 0.02%, 0.01%, or 0.005% self-condensed polyisocyanate, relative to the total weight of the microcapsule.

Preferably, the microcapsule shell of the present disclosure does not comprise a blend of biodegradable material and non-biodegradable material, that is, the microcapsule shell does not comprise a non-biodegradable material. In some embodiments, the microcapsule shell comprises a blend of biodegradable material and non-biodegradable material, but the level of the non-biodegradable material is no more than 10%, 5%, 3%, 1%, 0.5%, 0.2%, or 0.1% based on the total weight of the blend or the microcapsule shell. In some embodiments, the microcapsule shell comprises a blend of biodegradable material and non-biodegradable material, but the biodegradation rate of all components of the blend as a whole is at least 20%, 30%, 40%, 50%, 60%, 65%, 70%, 75%, 80%, 85%, 90%, 95% or 98%, based on the total weight of the blend, within 60 days according to OECD301F or OECD310.

The present disclosure also provides a process for preparing the biodegradable core-shell microcapsule. The process comprises (a) reacting, under an anhydrous condition, a polyisocyanate with a biopolymer and/or an amphiphilic compound, preferably in the presence of a catalyst, to form an isocyanate-functionalized prepolymer; (b) emulsifying the isocyanate-functionalized prepolymer with an aqueous solution to form an emulsion; (c) crosslinking the isocyanate-functionalized prepolymer and optionally the polyelectrolyte with a first crosslinker to form the biodegradable core-shell microcapsule, wherein the first crosslinker comprises an oxidized sugar comprising aldehyde groups and/or an enzyme selected from the group consisting of transglutaminase, laccase, peroxidase, oxidase, amylase, transferase, and mixtures thereof; (d) optionally further crosslinking the microcapsule shell with a second crosslinker selected from the group consisting of tannic acid, hydrolyzed tannic acid, tannin, gallic acid, methyl gallate, ethyl gallate, glutaraldehyde, glyoxal, triethyl citrate, malondialdehyde, genipin, dopamine, phenols, polyphenols, polycarbodiimide, polyacid chlorides, tetraethoxysilane, enzymes, multivalent cations, and mixtures thereof; and (e) optionally curing the microcapsule shell at a temperature ranging from 5° C. to 150° C. and at a pH ranging from 2 to 11; wherein the microcapsule shell has a biodegradation rate of at least 20%, 30%, 40%, 50%, 60%, 65%, 70%, 75%, 80%, 85%, 90%, 95% or 98%, based on the weight of the microcapsule shell, within 60 days according to OECD301F, and the microcapsule shell is substantially free of or free of a self-condensed polyisocyanate. In step (a), preferably the polyisocyanate is dissolved in a solution comprising a solvent and/or an active material. In step (b), preferably the aqueous solution comprises a polyelectrolyte.

Step (a) is the process of making the isocyanate-functionalized prepolymer as described in the present disclosure. The formed isocyanate-functionalized prepolymer is used in step (b). In some embodiments, the product mixture formed in step (a) is used directly in step (b) as an oil phase without purification or separation of the isocyanate-functionalized prepolymer from the product mixture. In step (b), an oil phase comprising the isocyanate-functionalized prepolymer is emulsified with an aqueous solution to form an emulsion. In some embodiments, the oil phase also comprises an active material. In some embodiments, the oil phase also comprises a solvent. Preferably, the active material and the solvent are the same ones as used in the process of making the isocyanate-functionalized prepolymer. In some embodiments, the aqueous solution comprises a polyelectrolyte. In some embodiments, the isocyanate-functionalized prepolymer and the polyelectrolyte form a coacervate.

In some embodiments, an emulsifier is used in step (b). In some embodiments, the emulsifier is selected from the group consisting of gelatin, collagen, chitosan, modified guar, modified glucan, gum Arabic (gum acacia), modified gum Arabic (modified gum acacia), proteins (e.g., pea protein), hydrolyzed proteins (e.g., hydrolyzed pea protein), fermented proteins, hydrophobin, enzymes, modified alginate, carrageenan, pectin, modified starch, modified cellulose, partially neutralized acid esters, polyvinyl alcohol, polystyrene sulfonates (e.g., Flexan II), co-polymers of ethylene and maleic anhydride (ZeMac), phospholipids, glycolipids, fatty acids, saponins, quillaia extract, surfactant salts with carboxylate, sulfate, sulfonate, phosphate, betaine, and/or linear alcohol groups, and mixtures thereof. Examples of emulsifiers also include particles for Pickering emulsions, such as cellulose particles, cyclodextrin particles, colloidal silica particles and/or quinoa particles. In some embodiments, the emulsifier comprises an amphiphilic compound selected from the group consisting of partially neutralized acid esters, polyvinyl alcohol, polystyrene sulfonates (e.g., Flexan II), co-polymers of ethylene and maleic anhydride (ZeMac), phospholipids, glycolipids, fatty acids, saponins, quillaia extract, surfactant salts with carboxylate, sulfate, sulfonate, phosphate, betaine, and/or linear alcohol groups, and mixtures thereof. In some embodiments, the emulsifier comprises a biopolymer selected from the group consisting of gelatin, collagen, chitosan, modified guar, modified glucan, gum Arabic (gum acacia), modified gum Arabic (modified gum acacia), proteins (e.g., pea protein), hydrolyzed proteins (e.g., hydrolyzed pea protein), fermented proteins, hydrophobin, enzymes, modified alginate, carrageenan, pectin, modified starch, modified cellulose, and combinations thereof. In some embodiments, the emulsifier comprises a gelatin. In some embodiments, the emulsifier comprises a pea protein, preferably non-denatured and/or non-hydrolyzed pea protein. In some embodiments, the emulsifier (e.g., gelatin, pea protein, and partially neutralized citric acid ester) can become part of the shell.

In some embodiments, the aqueous solution comprises a biopolymer or a polyelectrolyte which can function as an emulsifier. Such biopolymer or polyelectrolyte can be selected from the group consisting of gelatin, collagen, chitosan, modified guar, modified glucan, gum Arabic (gum acacia), modified gum Arabic (modified gum acacia), proteins (e.g., pea protein), hydrolyzed proteins (e.g., hydrolyzed pea protein), fermented proteins, hydrophobin, enzymes, partially neutralized citric acid ester, modified alginate, carrageenan, pectin, modified starch, modified cellulose, and combinations thereof. In some embodiments, such biopolymer or polyelectrolyte is selected from the group consisting of gelatin, chitosan, modified guar, modified glucan, gum Arabic, pea protein, and combinations thereof. In some embodiments, the biopolymer or polyelectrolyte is soluble or dispersible in the aqueous solution. In some embodiments, the emulsion is substantially free of or free of an emulsifier other than the biopolymer and polyelectrolyte. In some embodiments, the emulsion comprises no more than 0.5%, 0.2%, 0.1%, 0.05%, 0.02%, 0.01%, 0.005%, 0.002%, or 0.001%, based on the total weight of the emulsion, of an emulsifier other than the biopolymer and polyelectrolyte.

In some embodiments, in step (a), a polyisocyanate reacts with a biopolymer in the presence of a catalyst to form an isocyanate-functionalized prepolymer, the biopolymer comprises a chitosan, the polyelectrolyte used in step (b) comprises a gum Arabic, the first crosslinker used in step (c) comprises an oxidized sucrose, the second crosslinker used in step (d) comprises a tannic acid, and a pea protein is used in step (b) as an emulsifier. The chitosan prepolymer is crosslinked with oxidized sucrose and optionally tannic acid, gum Arabic is crosslinked with oxidized sucrose and tannic acid, and the chitosan prepolymer and gum Arabic are covalently bonded or crosslinked via the polyisocyanate and oxidized sucrose.

In some embodiments, the step (b) can be conducted at a temperature ranging from 20° C. to 150° C., or from 20° C. to 80° C., and at a pH ranging from 2 to 11, or from 2 to 6.5.

In some embodiments, the aqueous solution in step (b) comprises the first crosslinker. In some embodiments, the aqueous solution in step (b) comprises the first crosslinker and the polyelectrolyte. In some embodiments, the first crosslinker is added to the emulsion formed in step (b). In some embodiments, the first crosslinker is in the form of an aqueous solution. For example, a sugar and an oxidizing agent can be mixed (e.g., dissolved or dispersed) in an aqueous solution to form an oxidized sugar, and the resulting aqueous solution comprising the oxidized sugar can be added to the emulsion for crosslinking. In some embodiments, the sugar comprises sucrose. In some embodiments, the oxidizing agent comprises sodium periodate. In some embodiments, both the isocyanate-functionalized prepolymer and the polyelectrolyte are crosslinked with the first crosslinker to form a biodegradable core-shell microcapsule.

In some embodiments, the step (c) can be conducted at a temperature ranging from 20° C. to 150° C., or from 20° C. to 80° C., and at a pH ranging from 2 to 11, or from 2 to 6.5.

In some embodiments, the microcapsule shell is further crosslinked with a second crosslinker in step (d), that is, the isocyanate-functionalized prepolymer and/or the polyelectrolyte is further crosslinked with a second crosslinker. In some embodiments, the polyelectrolyte is further crosslinked with the second crosslinker. In some embodiments, both the isocyanate-functionalized prepolymer and the polyelectrolyte are further crosslinked with the second crosslinker. In some embodiments, the polyelectrolyte used in step (b) comprises an alginate, and the second crosslinker comprises a multivalent cation such as calcium ion. In some embodiments, the second crosslinker is added into the emulsion together with or after the addition of the first crosslinker. In some embodiments, the aqueous solution in step (b) comprises the first crosslinker and the second crosslinker. In some embodiments, the aqueous solution in step (b) comprises the first crosslinker, the second crosslinker and the polyelectrolyte.

In some embodiments, the step (d) can be conducted at a temperature ranging from 20° C. to 150° C., or from 20° C. to 80° C., and at a pH ranging from 2 to 11, or from 2 to 6.5.

In some embodiments, subsequent to the addition of the first crosslinker and the second crosslinker (if step (d) is performed), the microcapsule shell can be cured. In some embodiments, the curing step (e) can be performed before the addition of the second crosslinker (if step (d) is performed) and after the addition of the first crosslinker. In some embodiments, the curing step (e) can be performed before the addition of the first crosslinker and after the emulsifying step (b). The term “curing”, as used herein, means a toughening or hardening process of a polymer brought about by heat, chemical additives, and/or light radiation. In some embodiments, the microcapsule shell is cured at an elevated temperature. In some embodiments, the microcapsule shell is cured at a temperature ranging from 25° C. to 250° C., or from 20° C. to 150° C., or from 20° C. to 80° C., or from 40° C. to 85° C. In some embodiments, the microcapsule shell is cured for about 30 minutes to 24 hours, preferably 1 hour to 4 hours. In some embodiments, the microcapsule shell is cured at a pH ranging from 2 to 10, or from 2 to 6.5.

In some embodiments, the process may further comprise a step of drying the biodegradable core-shell microcapsule to remove water. The microcapsule can be dried at a temperature of from 20° C. to 250° C., or at room temperature. In some embodiments, the microcapsule is dried by a dehumidifier configured to supply desiccated air to the microcapsule, a radiant heat source for facilitating drying of the microcapsule, or submitting the microcapsule under a gas flow to obtain dried free-flowing microcapsule. It is understood that any standard method known by a person skilled in the art to perform such drying is also applicable.

Using the preparation process of the present disclosure, a relatively high encapsulation efficiency is achieved. The term “encapsulation efficiency” or “microencapsulation efficiency”, as used herein with respect to the preparation of microcapsule, means the amount (in weight) of the active material being encapsulated relative to the total amount (in weight) of the active material used in the preparation of the microcapsule. In accordance with the preparation process of the present disclosure, encapsulation efficiencies in the range of from 50% to 99.9% are attainable, or more preferably from 60% to 99.7%. In particular, encapsulation efficiencies of at least 90%, 92%, 94%, 96%, 98%, or 99% are achieved.

Another aspect of the present disclosure is the desire to move towards the use of fragrance ingredients and/or microcapsules derived from “Green Chemistry” principles. Green Chemistry is focused on the design of products and processes that minimize environmental impact, particularly by using renewable feedstocks. In other words, the raw material or feedstock used to make the fragrance ingredients and/or microcapsules should be sustainable rather than depleting whenever technically and economically practicable. Preferably, the fragrance components and/or microcapsules of the present disclosure have a bio-renewable carbon (BRC) content of at least 50%, at least 55%, at least 60%, at least 65%, at least 70%, at least 75%, at least 80%, at least 85%, at least 90% or at least 95%. As used herein, “BRC” refers to carbon that is part of the earth's natural environment and non-fossil-based carbon. BRC are naturally occurring renewable, repurposed and/or upcycled carbon resources that can be replenished to replace the portion depleted by usage and consumption, either through natural reproduction, or other recurring processes in a finite amount of time (such as within a human lifetime). BRC would exclude carbon that comes from virgin crude oil.

The biodegradable core-shell microcapsule of the present disclosure can be combined with one or more other delivery systems such as polymer-assisted delivery compositions (see U.S. Pat. No. 8,187,580), fiber-assisted delivery compositions (US 2010/0305021), cyclodextrin host-guest complexes (U.S. Pat. No. 6,287,603 and US 2002/0019369), pro-fragrances (WO 2000/072816 and EP 0 922 084), and any combination thereof. More exemplary delivery systems that can be incorporated are coacervate capsules, cyclodextrin delivery systems, and pro-perfumes.

Furthermore, microcapsules having one or more different characteristics can be combined to provide desirable or tailored release profiles and/or stability. In particular, the microcapsule slurry can include a combination of two or more types of microcapsules that differ in their encapsulating wall materials, microcapsule size, amounts of wall materials, the thickness of the wall, the degree of polymerization, the degree of crosslinking, ratios between the wall materials and the active material, core modifiers, scavengers, active materials, cure temperatures, heating rates during the curing, curing times, the rupture force or fracture strength, or a combination thereof. In some aspects, the microcapsule slurry is composed of two, three, four, five, six, seven or more different types of capsules that differ by one or more of the above-referenced characteristics.

When assessing storage stability, fragrance retention within the microcapsule may be measured directly after storage, at a desired temperature and different time periods such as four weeks, six weeks, two months, three months or more in a consumer product base. The preferred manner is to measure total headspace of the consumer product at the specified time and to compare the results to the headspace of a control consumer product made to represent 0% retention via direct addition of the total amount of fragrance present. Alternatively, the consumer product may be performance tested after the storage period and the performance compared to the fresh product, either analytically or by sensory evaluation. This measurement often involves either measuring the fragrance headspace over a substrate used with the product, or odor evaluation of the same substrate.

In certain aspects, retention of the active material in the core of the microcapsule is assessed in a consumer product, e.g., under storage conditions such as at a temperature in the range of from 25° C. to 40° C., or more preferably in the range of from 30° C. to 37° C., or most preferably 37° C., for an extended period of time of at least 2 weeks, 4 weeks, 6 weeks, 8 weeks, 16 weeks, or 32 weeks. In certain aspects, the microcapsule of the present disclosure retains at least 30%, 40%, 45%, 50%, 55%, 60%, 65%, 70%, 75%, 80%, 85%, 90%, 95%, or 99% of the active material when added to a consumer product and stored under the storage conditions and for the extended period of time as described above. In particular aspects, the microcapsule of the present disclosure, when added to a consumer product, retains between 40% and 90% of the active material after being stored at 37° C. for at least 4 weeks, 8 weeks or 12 weeks. Alternatively stated, the microcapsule of the present disclosure loses less than 50% of the active material due to leakage when added to a consumer product and stored for 8 weeks at 37° C.

The biodegradable core-shell microcapsule of the present disclosure is well-suited for inclusion in any of a variety of consumer products where controlled release of active material (e.g., fragrances or flavors) is desired. The microcapsule of the present disclosure can be added to a consumer product directly or be printed onto a product base or a movable product conveyor (e.g., a non-stick belt) for drying. See WO 2019/212896 A1. The biodegradable core-shell microcapsule can be added to the consumer product at a level in the range of from 0.001% to 50%, or preferably from 0.01% to 50% by weight of the consumer product. In particular, the biodegradable core-shell microcapsule is suitably included in a consumer product such as a pharmaceutical, agricultural or cosmetic formulation. Examples of consumer products include, but are not limited to, a fabric softener, fabric conditioner, detergent, scent booster, fabric refresher spray, body wash, body soap, shampoo, hair conditioner, body spray, hair refresher spray, hair dye, hair moisturizer, skin moisturizer, hair treatment, antiperspirant, deodorant, skin treatment, insect repellant, candle, surface cleaner, bathroom cleaner, bleach, cat litter, refresher spray, pesticide, insecticide, herbicide, fungicide, or paint.

The values and dimensions disclosed herein are not to be understood as being strictly limited to the exact numerical values recited. Instead, unless otherwise specified, each such value is intended to mean both the recited value and a functionally equivalent range surrounding that value. For example, a value disclosed as “50%” is intended to mean “about 50%.”

### Applications

The delivery systems of the present disclosure are well-suited for use, without limitation, in a laundry detergent, a liquid laundry detergent, a powder laundry detergent, a tablet laundry detergent, a laundry detergent bar, a laundry detergent cream, a hand wash laundry detergent, a fabric conditioner or softener, a fabric refresher, a scent booster, a shampoo, a hair conditioner, a bar soap, a shower gel, a body wash, an antiperspirant, a body spray, a body mist, a lotion, a candle or a textile.

More specifically, the microcapsules of the present disclosure can be used in the following products:

A) Fabric Care Products such as Rinse Conditioners (containing 1 to 30 weight % of a fabric conditioning active), Fabric Liquid Conditioners (containing 1 to 30 weight % of a fabric conditioning active), Tumble Drier Sheets, Fabric Refreshers, Fabric Refresher Sprays, Ironing Liquids, and Fabric Softener Systems such as those described in U.S. Pat. Nos. 6,335,315, 5,674,832, 5,759,990, 5,877,145, 5,574,179, 5,562,849, 5,545,350, 5,545,340, 5,411,671, 5,403,499, 5,288,417, 4,767,547 and 4,424,134.

Liquid fabric softeners/fresheners contain at least one fabric softening agent present, preferably at a concentration of 1 to 30% (e.g., 4% to 20%, 4% to 10%, and 8% to 15%) by weight of the liquid fabric softener/freshener. The ratio between the active material and the fabric softening agent can be 1:500 to 1:2 (e.g., 1:250 to 1:4 and 1:100 to 1:8). As an illustration, when the fabric softening agent is 5% by weight of the fabric softener, the active material is 0.01% to 2.5%, preferably 0.02% to 1.25% and more preferably 0.1% to 0.63%. As another example, when the fabric softening agent is 20% by weight of the fabric softener, the active material is 0.04% to 10%, preferably 0.08% to 5% and more preferably 0.4% to 2.5%. The active material is a fragrance, malodor counteractant or a combination thereof. The liquid fabric softener can have 0.15% to 15% of capsules (e.g., 0.5% to 10%, 0.7% to 5%, and 1% to 3%). When including capsules at these levels, the neat oil equivalent (NOE) in the softener is 0.05% to 5% (e.g., 0.15% to 3.2%, 0.25% to 2%, and 0.3% to 1%).

Suitable fabric softening agents include cationic surfactants. Non-limiting examples are quaternary ammonium compounds (QAC) such as alkylated quaternary ammonium compounds, ring or cyclic quaternary ammonium compounds, aromatic quaternary ammonium compounds, diquaternary ammonium compounds, alkoxylated quaternary ammonium compounds, amidoamine quaternary ammonium compounds, ester quaternary ammonium compounds, or a combination thereof.

Fabric softening product includes an aqueous QAC which are characterized by:
a) The viscosity of the final product ranges from 5 to 300 cps @ 106s−1, preferable 20 to 150 cps;
b) The level of QAC ranges 0.5 to 20 wt %, preferably from 1 to 16 wt %, more preferably 6 to 12 wt % softening active. The preferred, typical cationic fabric softening components include water-insoluble quaternary-ammonium fabric softeners, the most commonly used having been di-long alkyl chain ammonium chloride or methyl sulfate. Preferred cationic softeners include but not limited to the following:
a. rapidly biodegradable quaternary ammonium compounds which contain 1 or more ester bonds situated between the quaternary-ammonium group and the long alkyl chain (e.g., TEA ester quats, DEEDMAC and HEQ);
b. Non-Ester quaternary ammonium compounds (e.g., ditallow dimethylammonium chloride (DTDMAC); dihydrogenated tallow dimethylammonium chloride; dihydrogenated tallow dimethylammonium methylsulfate; distearyl dimethylammonium chloride; dioleyl dimethylammonium chloride; dipalmityl hydroxyethyl methylammonium chloride; stearyl benzyl dimethylammonium chloride; tallow trimethylammonium chloride; hydrogenated tallow trimethylammonium chloride; C12-14 alkyl hydroxyethyl dimethylammonium chloride; C12-18 alkyl dihydroxyethyl methylammonium chloride; di(stearoyloxyethyl) dimethylammonium chloride (DSOEDMAC); di(tallowoyloxyethyl) dimethylammonium chloride; ditallow imidazolinium methylsulfate; 1-(2-tallowylamidoethyl)-2-tallowyl imidazolinium methylsulfate.

A first group of quaternary ammonium compounds (QACs) suitable for use according to the present disclosure is represented by formula (I):

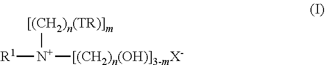

wherein each R is independently selected from a C₁-C₃₅ alkyl or alkenyl group; R¹ represents a C₁-C₄ alkyl, C₂-C₄ alkenyl or a C₁-C₄ hydroxyalkyl group; T is generally O—CO (i.e., an ester group bound to R via its carbon atom), but may alternatively be CO—O (i.e., an ester group bound to R via its oxygen atom); n is a number selected from 1 to 4; m is a number selected from 1, 2, or 3; and X is an anionic counter-ion, such as a halide or alkyl sulphate, e.g., chloride or methylsulphate. Di-esters variants of formula (I) (i.e., m=2) are preferred and typically have mono- and tri-ester analogues associated with them.

Especially preferred agents are preparations which are rich in the di-esters of triethanolammonium methylsulfate, otherwise referred to as “TEA ester quats”. Commercial examples include STEPANTEX® UL85, ex Stepan, Prapagen™ TQL, ex Clariant, and Tetranyl™ AHT-1, ex Kao, (both di-[hardened tallow ester] of triethanolammonium methylsulphate), AT-1 (di-[tallow ester] of triethanolammonium methylsulphate), and L5/90 (di-[palm ester] of triethanolammonium methylsulphate), both ex Kao, and REWOQUAT® WE15 (a di-ester of triethanolammonium methylsulphate having fatty acyl residues deriving from C10-C₂₀ and C₁₆-C₁₈ unsaturated fatty acids), ex Evonik.

Also suitable are soft quaternary ammonium actives such as STEPANTEX® VK90, STEPANTEX® VT90, SP88 (ex-Stepan), Prapagen™ TQ (ex-Clariant), DEHYQUART® AU-57 (ex-Cognis), REWOQUAT® WE18 (ex-Degussa) and Tetranyl™ L190 P, Tetranyl™ L190 SP and Tetranyl™ L190 S (all ex-Kao).

A second group of QACs suitable for use according to the present disclosure is represented by formula (II):

(R¹)₂—N⁺—[(CH₂)ₙ-T-R²]₂X⁻ (II)

wherein each R¹ group is independently selected from C₁-C₄ alkyl, or C₂-C₄ alkenyl groups; and wherein each R² group is independently selected from C₈-C₂₈ alkyl or alkenyl groups; and n, T, and X—are as defined above. Preferred materials of this second group include bis(2tallwoyloxyethyl)dimethyl ammonium chloride and hardened versions thereof.

A third group of QACs suitable for use according to the present disclosure is represented by formula (III):

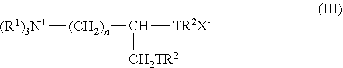

wherein each R¹ group is independently selected from C₁-C₄ alkyl, hydroxyalkyl or C₂-C₄ alkenyl groups; and wherein each R² group is independently selected from C₈-C₂₈ alkyl or alkenyl groups; and wherein n, T, and X are as defined above. Preferred materials of this second group include 1,2 bis[tallowoyloxy]-3-trimethylammonium propane chloride, 1,2 bis[hardened tallowoyloxy]-3-trimethylammonium propane chloride, 1,2-bis[oleoyloxy]-3 trimethylammonium propane chloride, and 1,2 bis[stearoyloxy]-3-trimethylammonium propane chloride. Such materials are described in U.S. Pat. No. 4,137,180 (Lever Brothers). Preferably, these materials also comprise an amount of the corresponding mono-ester.

Co-softeners. Co-softeners, also referred to as co-softeners and fatty complexing agents may be used in fabric conditioner composition of the present disclosure. When employed, they are typically present at from 0.1 to 20% and particularly at from 0.1 to 5%, based on the total weight of the composition. Preferred co-softeners include fatty alcohols, fatty esters, and fatty N-oxides. Fatty esters that may be employed include fatty monoesters, such as glycerol monostearate, fatty sugar esters, such as those disclosed WO 01/46361 (Unilever).

In some embodiments, the compositions of the present disclosure may comprise a co-actives. Especially suitable fatty complexing agents include fatty alcohols and fatty acids. Of these, fatty alcohols are most preferred. Without being bound by theory it is believed that the fatty complexing material improves the viscosity profile of the composition by complexing with mono-ester component of the fabric conditioner material thereby providing a composition which has relatively higher levels of di-ester and tri-ester linked components. The di-ester and tri-ester linked components are more stable and do not affect initial viscosity as detrimentally as the mono-ester component. It is also believed that the higher levels of mono-ester linked component present in compositions comprising quaternary ammonium materials based on TEA may destabilize the composition through depletion flocculation. By using the co-active material to complex with the mono-ester linked component, depletion flocculation is significantly reduced. In other words, the co-active at the increased levels, as required by the present disclosure in some embodiments, “neutralizes” the mono-ester linked component of the quaternary ammonium material. This in situ di-ester generation from mono-ester and fatty alcohol also improves the softening of the composition.

Silicone. In some embodiments, the compositions of the present disclosure may further contain a silicone based fabric softening agent. Preferably the fabric softening silicone is a polydimethylsiloxane. The fabric softening silicones include but are not limited to 1) non-functionalized silicones such as polydimethylsiloxane (PDMS) or alkyl (or alkoxy) functional silicones; 2) functionalized silicones or copolymers with one or more different types of functional groups such as amino, phenyl, polyether, acrylate, silicon hydride, carboxylic acid, quaternized nitrogen, etc. Suitable silicones may be selected from polydialkylsiloxanes, preferably polydimethylsiloxane more preferably amino functionalised silicones; anionic silicones and carboxyl functionalized silicone. An amino silicone that may also be used, for example, Arristan 64, ex CHT or Wacker CT45E, ex Wacker.

In terms of silicone emulsions, the particle size can be in the range from about 1 nm to 100 microns and preferably from about 10 nm to about 10 microns including microemulsions (<150 nm), standard emulsions (about 200 nm to about 500 nm) and macroemulsions (about 1 micron to about 20 microns).

Non-ionic surfactants. In some embodiments, the compositions may further comprise a nonionic surfactant. Typically, these can be included for the purpose of stabilizing the compositions. Suitable nonionic surfactants include addition products of ethylene oxide with fatty alcohols, fatty acids, and fatty amines. Any of the alkoxylated materials of the particular type described hereinafter can be used as the nonionic surfactant. Suitable surfactants are substantially water soluble surfactants of the general formula (V): R—Y—(C₂H₄O)z-CH₂—CH₂—OH (V) where R is selected from the group consisting of primary, secondary and branched chain alkyl and/or acyl hydrocarbyl groups; primary, secondary and branched chain alkenyl hydrocarbyl groups; and primary, secondary and branched chain alkenyl-substituted phenolic hydrocarbyl groups; the hydrocarbyl groups having a chain length of from 8 to about 25, preferably 10 to 20, e.g., 14 to 18 carbon atoms. In the general formula for the ethoxylated nonionic surfactant, Y is typically: —O—, —C(O)O—, —C(O)N(R)— or —C(O)N(R)R in which R has the meaning given above for formula (V), or can be hydrogen; and Z is at least about 8, preferably at least about 10 or 11.

Preferably the nonionic surfactant has an HLB of from about 7 to about 20, more preferably from 10 to 18, e.g., 12 to 16. GENAPOL® C200 (Clariant) based on coco chain and 20 EO groups is an example of a suitable nonionic surfactant. If present, the nonionic surfactant is present in an amount from 0.01 to 10%, more preferably 0.1 to 5 by weight, based on the total weight of the composition. LUTENSOL® AT25 (BASF) based on coco chain and 25 EO groups is an example of a suitable non-ionic surfactant. Other suitable surfactants include RENEX® 36 (Trideceth-6), ex Croda; TERGITOL® 15-S3, ex Dow Chemical Co.; Dihydrol LT7, ex Thai Ethoxylate ltd; CREMOPHOR® CO40, ex BASF and NEODOL® 91-8, ex Shell.

Cationic Polysaccharide. In some embodiments, the compositions may further comprise at least one cationic polysaccharide. The cationic polysaccharide can be obtained by chemically modifying polysaccharides, generally natural polysaccharides. By such modification, cationic side groups can be introduced into the polysaccharide backbone The cationic polysaccharides are not limited to: cationic cellulose and derivatives thereof, cationic starch and derivatives thereof, cationic callose and derivatives thereof, cationic xylan and derivatives thereof, cationic mannan and derivatives thereof, cationic galactomannan and derivatives thereof, such as cationic guar and derivatives thereof. Cationic celluloses which are suitable include cellulose ethers comprising quaternary ammonium groups, cationic cellulose copolymers or celluloses grafted with a water-soluble quaternary ammonium monomer.

The cellulose ethers comprising quaternary ammonium groups are described in French patent 1,492,597 and in particular include the polymers sold under the names “JR” (JR 400, JR 125, JR 30M) or “LR” (LR 400, LR 30M) by the company Dow. These polymers are also defined in the CTFA dictionary as hydroxyethylcellulose quaternary ammoniums that have reacted with an epoxide substituted with a trimethylammonium group. Suitable cationic celluloses also include LR3000 KC from Solvay. The cationic cellulose copolymers or the celluloses grafted with a water-soluble quaternary ammonium monomer are described especially in patent U.S. Pat. No. 4,131,576, such as hydroxyalkylcelluloses, for instance hydroxymethyl-, hydroxyethyl- or hydroxypropylcelluloses grafted especially with a methacryloyl-ethyltrimethylammonium, methacrylamidopropyltrimethylammonium or dimethyl-diallylammonium salt.

The commercial products corresponding to this definition are more particularly the products sold under the names CELQUAT® L 200 and CELQUAT® H 100 by Akzo Nobel. Cationic starches suitable for the present disclosure include the products sold under POLYGELO® (cationic starches from Sigma), the products sold under SOFTGEL®, AMYLOFAX® and SOLVITOSE® (cationic starches from Avebe), CATO from National Starch. Suitable cationic galactomannans can be those derived from Fenugreek Gum, Konjac Gum, Tara Gum, Cassia Gum or Guar Gum.

In some embodiments, the cationic polysaccharide of the present disclosure may have an average Molecular Weight (Mw) of between 100,000 daltons and 3,500,000 daltons, preferably between 100,000 daltons and 1,500,000 daltons, more preferably between 100,000 daltons and 1,000,000 daltons.

In some embodiments, the fabric conditioner composition of the present disclosure preferably comprises from 0.01 to 2 wt % of cationic polysaccharide based on the total weight of the composition. More preferably, 0.025 to 1 wt % of cationic polysaccharide based on the total weight of the composition. Most preferably, 0.04 to 0.8 wt % of cationic polysaccharide based on the total weight of the composition. In the context of the present application, the term “Degree of Substitution (DS)” of cationic polysaccharides, such as cationic guars, is the average number of hydroxyl groups substituted per sugar unit. DS may notably represent the number of the carboxymethyl groups per sugar unit. DS may be determined by titration.

The DS of the cationic polysaccharide is preferably in the range of 0.01 to 1, more preferably 0.05 to 1, most preferably 0.05 to 0.2. In the context of the present application, “Charge Density (CD)” of cationic polysaccharides, such as cationic guars, means the ratio of the number of positive charges on a monomeric unit of which a polymer is comprised to the molecular weight of said monomeric unit. CD of the cationic polysaccharide, such as the cationic guar, is preferably in the range of 0.1 to 3 (meq/gm), more preferably 0.1 to 2 (meq/gm), most preferably 0.1 to 1 (meq/gm).

Non-ionic Polysaccharide. In some embodiments, the fabric conditioner composition may further comprise at least one non-ionic polysaccharide. The nonionic polysaccharide can be a modified nonionic polysaccharide or a non-modified nonionic polysaccharide. The modified non-ionic polysaccharide may comprise hydroxyalkylation and/or esterification. In the context of the present disclosure, the level of modification of non-ionic polysaccharides can be characterized by Molar Substitution (MS), which means the average number of moles of substituents, such as hydroxypropyl groups, per mole of the monosaccharide unit. MS can be determined by the Zeisel-GC method, notably based on the following literature reference: Hodges, et al. (1979) Anal. Chem. 51 (13). Preferably, the MS of the modified nonionic polysaccharide is in the range of 0 to 3, more preferably 0.1 to 3 and most preferably 0.1 to 2.

In some embodiments, the nonionic polysaccharide of the present disclosure may be especially chosen from glucans, modified or non-modified starches (such as those derived, for example, from cereals, for instance wheat, corn or rice, from vegetables, for instance yellow pea, and tubers, for instance potato or cassava), amylose, amylopectin, glycogen, dextrans, celluloses and derivatives thereof (methylcelluloses, hydroxyalkylcelluloses, ethylhydroxyethylcelluloses), mannans, xylans, lignins, arabans, galactans, galacturonans, chitin, chitosans, glucuronoxylans, arabinoxylans, xyloglucans, glucomannans, pectic acids and pectins, arabinogalactans, carrageenans, agars, gum Arabics, gum tragacanths, ghatti gums, karaya gums, carob gums, galactomannans such as guars and nonionic derivatives thereof (hydroxypropyl guar), and mixtures thereof.

Among the celluloses that can be especially used are hydroxyethylcelluloses and hydroxypropylcelluloses. Suitable non-limiting examples include products sold under the trade names KLUCEL® EF, KLUCEL® H, KLUCEL® LHF, KLUCEL® MF and KLUCEL® G by Aqualon, and CELLOSIZE® Polymer PCG-10 by Amerchol, and HEC, HPMC K200, HPMC K35M by Ashland.

In some embodiments, the fabric conditioner composition of the present disclosure preferably comprises from 0.01 to 2 wt % of non-ionic polysaccharide based on the total weight of the composition. More preferably, 0.025 to 1 wt % of non-ionic polysaccharide based on the total weight of the composition. Most preferably, 0.04 to 0.8 wt % of non-ionic polysaccharide based on the total weight of the composition. Preferably the fabric conditioning composition comprises combined weight of the cationic polysaccharide and non-ionic polysaccharide of 0.02 to 4 wt %, more preferably 0.05 to 2 wt % and most preferably 0.08 to 1.6 wt %. Preferably the ratio of the weight of the cationic polysaccharide in the composition and the weight of the nonionic polysaccharide in the composition is between 1:10 and 10:1, more preferably, between 1:3 and 3:1.

In a preferred embodiment, the cationic polysaccharide and non-ionic polysaccharide are mixed prior to addition to the fabric conditioner composition. Preferably the mix is prepared as a suspension in water. Preferably, the ratio of the weight of the quaternary ammonium compound in the composition and the total weight of the cationic polysaccharide and the nonionic polysaccharide in the composition is between 100:1 and 2:1, more preferably, between 30:1 and 5:1.

Water. In some embodiments, the fabric conditioner composition of the present disclosure comprises water. The compositions are rinse-added softening compositions suitable for use in a laundry process. The compositions are pourable liquids. The liquid compositions have a pH ranging from about 2.0 to about 7, preferably from about 2 to about 4, more preferably from about 2.5 to about 3.5. The compositions may also contain pH modifiers preferably hydrochloric acid, lactic acid or sodium hydroxide. The composition is preferably a ready-to-use liquid comprising an aqueous phase. The aqueous phase may comprise water-soluble species, such as mineral salts or short chain (C₁-C₄) alcohols. The composition is preferably for use in the rinse cycle of a home textile laundering operation, where, it may be added directly in an undiluted state to a washing machine, e.g., through a dispenser drawer or, for a top-loading washing machine, directly into the drum. The compositions may also be used in a domestic hand-washing laundry operation.

The fabric conditioner composition may typically be made by combining a melt comprising the fabric softening agent with an aqueous phase. The polymer may be combined with the water phase, or it may be post dosed into the composition after combination of the melt and water phase. A preferred method of preparation is as follows:
1. Heat water to about 40 to 50° C., preferably above 45° C.
2. Add the rheology modifiers to the water slowly, preferably over about 1 minute with stirring.
3. Mix thoroughly, preferably from 1 to 10 minutes.
4. Add any minor ingredients, such as antifoams, sequestrants and preservatives.
5. Melt the softening active and optional fatty alcohol together to form a co-melt.
6. Add the co-melt to the heated water.
7. Add acid to the preferred pH, if required.
8. Add dyes and perfumes.
9. Cool.

B) Liquid dish detergents such as those described in U.S. Pat. Nos. 6,069,122 and 5,990,065.

C) Automatic Dish Detergents such as those described in U.S. Pat. Nos. 6,020,294, 6,017,871, 5,968,881, 5,962,386, 5,939,373, 5,914,307, 5,902,781, 5,705,464, 5,703,034, 5,703,030, 5,679,630, 5,597,936, 5,581,005, 5,559,261, 4,515,705, 5,169,552, and 4,714,562.

D) All-purpose cleaners including bucket dilutable cleaners and toilet cleaners, bathroom cleaners, bath tissue, rug deodorizers, candles (e.g., scented candles), room deodorizers, floor cleaners, disinfectants, window cleaners, garbage bags/trash can liners, air fresheners (e.g., room deodorizer, car deodorizer, sprays, scent oil air freshener, automatic spray air freshener, and neutralizing gel beads), moisture absorber, household devices (e.g., paper towels and disposable wipes), and moth balls/traps/cakes.

E) Personal care products: cosmetic or pharmaceutical preparations. More specifically personal cleansers (e.g., bar soaps, body washes, and shower gels), in-shower conditioner, sunscreen (e.g., sprays, lotions and sticks), insect repellents, hand sanitizers, anti-inflammatory (e.g., balms, ointments and sprays), antibacterial (e.g., ointments and creams), sensates, deodorants and antiperspirants (including aerosol, pump spray and wax based), lotions, body powder and foot powder, body mist or body spray, shave cream and male groom products, bath soak, exfoliating scrub.

F) Hair Care products. More specifically, shampoos (liquid and dry powder), hair conditioners (rinse-out conditioners, leave-in conditioners, and cleansing conditioners), hair rinses, hair refreshers, hair perfumes, hair straightening products, hair styling products, hair fixative and styling aids, hair combing creams, hair wax, hair foam, hair gel, non-aerosol pump spray, hair bleaches, dyes and colorants, perming agents, and hair wipes.

In particular aspects, the core-shell microcapsule slurry of this disclosure is of use in improving a freshness impression to a fabric. Accordingly, in certain aspects, the microcapsules of the present disclosure are included in a fabric conditioner or softener having a pH of from 2 to 4, preferably a pH of from 2.5 to 3.5.

Many aspects and embodiments have been described above and are merely exemplary and not limiting. After reading this specification, skilled artisans appreciate that other aspects and embodiments are possible without departing from the scope of the invention.

### EXAMPLES

The following non-limiting examples are provided to further illustrate the present invention and are not to be construed as limitations of the invention, as many variations of the present invention are possible without departing from its spirit or scope.

### General

Unless otherwise specified, Magnitude is a fragrance commercially available from International Flavors & Fragrances Inc., NY; Zazu is a fragrance commercially available from International Flavors & Fragrances Inc., NY; Mermaid is a fragrance commercially available from International Flavors & Fragrances Inc., NY; caprylic/capric triglyceride used in the Examples is the commercial product under the tradename NEOBEE® M-5 from Stepan in Chicago, IL; and the polyisocyanate used in the Examples is the commercial product under the tradename TAKENATE® D-110N from Mitsui Chemicals in Japan. A person of ordinary skill in the art appreciates that some polyisocyanate commercial products is a solution of polyisocyanate in a solvent. The polyisocyanate (e.g., TAKENATE® D-110N) amount indicated in this disclosure means the amount of polyisocyanate itself, that is, the amount of the solvent is excluded.

### Example 1: Gelatin Prepolymer Microcapsule

An oil phase was prepared by mixing Magnitude fragrance, caprylic/capric triglyceride (NEOBEE® M-5) and polyisocyanate (TAKENATE® D-110N). Gelatin was subsequently dispersed in the oil phase under constant mixing. The oil phase mixture was heated with constant mixing and 1,4-diazabicyclo[2.2.2]octane (DABCO) was optionally added to catalyze prepolymer formation for the reaction times and temperatures indicated in Tables 3-6. Separately, an aqueous solution of gum Arabic was prepared and then emulsified with the oil phase at 6000 rpm for 3 minutes to form an emulsion. After allowing the emulsion to stir at 40° C. for 1 hour, the pH of the emulsion was gradually adjusted to 3.5 using 50% citric acid. An aqueous crosslinking solution containing mixture of sucrose and sodium periodate was prepared and stirred at room temperature for 30 minutes. The aqueous crosslinking solution was then added to the emulsion and stirring was maintained at 40° C. for 4 hours. The resulting microcapsule slurries were designated Microcapsules 1-14.

TABLE 3

Microcapsule (%)¹

| | 1 | 2 | 3 |
|---|---|---|---|
| Microcapsule Component | | | |
| Water | 82% | 82% | 82% |
| Gum Arabic | 2.0% | 2.0% | 2.0% |
| Gelatin | 1.5% | 1.5% | 1.5% |
| Magnitude Fragrance | 10% | 10% | 10% |
| NEOBEE ® M-5 | 2.5% | 2.5% | 2.5% |
| TAKENATE ® D-110N | 0.25% | 0.25% | 0.25% |
| Citric Acid | 0.30% | 0.30% | 0.30% |
| Sucrose | 1.0% | 1.0% | 1.0% |
| Sodium Periodate | 0.10% | 0.10% | 0.10% |
| DABCO | 0.015% | 0.015% | 0.015% |
| Prepolymer Formation² | | | |
| Temperature (° C.) | 60° C. | 60° C. | 60° C. |
| Reaction Time (minutes) | 10 | 30 | 60 |

¹Percentages are based on total weight of the microcapsule slurry.
²Reaction temperature and time for forming the isocyanate-functionalized prepolymer.

TABLE 4

Microcapsule (%)¹

| | 4 | 5 | 6 |
|---|---|---|---|
| Microcapsule Component | | | |
| Water | 82% | 82% | 82% |
| Gum Arabic | 2.0% | 2.0% | 2.0% |
| Gelatin | 1.5% | 1.5% | 1.5% |
| Magnitude Fragrance | 10% | 10% | 10% |
| NEOBEE ® M-5 | 2.5% | 2.5% | 2.5% |
| TAKENATE ® D-110N | 0.25% | 0.25% | 0.25% |
| Citric Acid | 0.30% | 0.30% | 0.30% |
| Sucrose | 1.0% | 1.0% | 1.0% |
| Sodium Periodate | 0.10% | 0.10% | 0.10% |
| DABCO | 0.015% | 0.015% | 0.015% |
| Prepolymer Formation³ | | | |
| Temperature (° C.) | RT² | RT² | RT² |
| Reaction Time (minutes) | 10 | 30 | 60 |

¹Percentages are based on total weight of the microcapsule slurry.
²RT, room temperature.
³Reaction temperature and time for forming the isocyanate-functionalized prepolymer.

TABLE 5

| | Microcapsule (%)¹ | | | |
|---|---|---|---|---|
| | 7 | 8 | 9 | 10 |
| Microcapsule Component | | | | |
| Water | 82% | 82% | 82% | 82% |
| Gum Arabic | 2.0% | 2.0% | 2.0% | 2.0% |
| Gelatin | 1.5% | 1.5% | 1.5% | 1.5% |
| Magnitude Fragrance | 10% | 10% | 10% | 10% |
| NEOBEE ® M-5 | 2.5% | 2.5% | 2.5% | 2.5% |
| TAKENATE ® D-110N | 0.25% | 0.25% | 0.25% | 0.25% |
| Citric Acid | 0.30% | 0.30% | 0.30% | 0.30% |
| Sucrose | 1.0% | 1.0% | 1.0% | 1.0% |
| Sodium Periodate | 0.10% | 0.10% | 0.10% | 0.10% |
| DABCO | 0.10% | 0.10% | 0.10% | 0.10% |
| Prepolymer Formation³ | | | | |
| Temperature (° C.) | 60° C. | 60° C. | RT² | RT² |
| Reaction Time (minutes) | 10 | 60 | 10 | 60 |

¹Percentages are based on total weight of the microcapsule slurry.
²RT, room temperature.
³Reaction temperature and time for forming the isocyanate-functionalized prepolymer.

TABLE 6

| | Microcapsule (%)¹ | | | |
|---|---|---|---|---|
| | 11 | 12 | 13 | 14 |
| Microcapsule Component | | | | |
| Water | 82% | 82% | 82% | 82% |
| Gum Arabic | 2.0% | 2.0% | 2.0% | 2.0% |
| Gelatin | 1.5% | 1.5% | 1.5% | 1.5% |
| Magnitude Fragrance | 10% | 10% | 10% | 10% |
| NEOBEE ® M-5 | 2.5% | 2.5% | 2.5% | 2.5% |
| TAKENATE ® D-110N | 0.25% | 0.25% | 0.25% | 0.25% |
| Citric Acid | 0.30% | 0.30% | 0.30% | 0.30% |
| Sucrose | 1.0% | 1.0% | 1.0% | 1.0% |
| Sodium Periodate | 0.10% | 0.10% | 0.10% | 0.10% |
| DABCO | 0.00% | 0.00% | 0.00% | 0.00% |
| Prepolymer Formation³ | | | | |
| Temperature (° C.) | 60° C. | 60° C. | RT² | RT² |
| Reaction Time (minutes) | 10 | 60 | 10 | 60 |

¹Percentages are based on total weight of the microcapsule slurry.
²RT, room temperature.
³Reaction temperature and time for forming the isocyanate-functionalized prepolymer.

The encapsulation efficiency (EE) of microcapsule slurries 1-14 was obtained and is presented in Table 7.

TABLE 7

| Microcapsule | EE |
|---|---|
| 1 | 99% |
| 2 | 99% |
| 3 | 86% |
| 4 | 99% |
| 5 | 99% |
| 6 | 99% |
| 7 | 75% |
| 8 | 18% |
| 9 | 99% |
| 10 | 20% |
| 11 | 99% |
| 12 | 99% |
| 13 | 99% |
| 14 | 99% |

### Example 2: Conversion of Gelatin and Polyisocyanate in Oil Phase to Form Prepolymer

An oil phase was prepared by mixing benzyl benzoate and polyisocyanate (TAKENATE® D-110N). Gelatin was subsequently dispersed in the oil phase under constant mixing. The oil phase mixture was heated with constant mixing and DABCO was optionally added to catalyze prepolymer formation for the reaction times and temperatures indicated in Tables 8a-8c. The formed prepolymers are designated Prepolymers 1-14 in Tables 8a-8c. Similarly, three additional prepolymer samples were prepared under the reaction times and temperatures of Prepolymers 1A-3A shown in Table 8a. The formed three additional prepolymers are designated Prepolymers 1A-3A in Table 8a. In Prepolymers 1A-3A, gelatin was omitted to demonstrate that the polyisocyanate conversion in Prepolymers 1-14 was mainly due to the presence of gelatin. At the end of the prepolymer preparations of Prepolymers 1-14 and 1A-3A, a 50 mg aliquot of each oil phase reaction mixture was taken and quenched with 7.9 g of methanol. These quenched reaction mixture samples were then analyzed for methyl urethane adduct by liquid chromatography (LC) to determine the residual amount of polyisocyanate in the reaction mixture. Polyisocyanate conversions in Prepolymers 1-14 and 1A-3A are presented in Table 8d.

TABLE 8a

| | Prepolymer (%)¹ | | | | | |
|---|---|---|---|---|---|---|
| | 1 | 2 | 3 | 1A | 2A | 3A |
| Reaction Mixture | | | | | | |
| Gelatin | 12.5% | 12.5% | 12.5% | 0.0% | 0.0% | 0.0% |
| Benzyl Benzoate | 85.3% | 85.3% | 85.3% | 85.3% | 85.3% | 85.3% |
| TAKENATE ® D-110N | 2.1% | 2.1% | 2.1% | 2.1% | 2.1% | 2.1% |
| DABCO | 0.1% | 0.1% | 0.1% | 0.1% | 0.1% | 0.1% |
| Reaction Condition | | | | | | |
| Temperature (° C.) | 60° C. | 60° C. | 60° C. | 60° C. | 60° C. | 60° C. |
| Reaction Time (minutes) | 10 | 30 | 60 | 10 | 30 | 60 |

¹Percentages are based on total weight of the reaction mixture.

TABLE 8b

| | Prepolymer (%)¹ | | | | | |
|---|---|---|---|---|---|---|
| | 4 | 5 | 6 | 7 | 8 | 9 |
| Reaction Mixture | | | | | | |
| Gelatin | 12.5% | 12.5% | 12.5% | 12.4% | 12.4% | 12.4% |
| Benzyl Benzoate | 85.3% | 85.3% | 85.3% | 84.7% | 84.7% | 84.7% |
| TAKENATE ® D-110N | 2.1% | 2.1% | 2.1% | 2.1% | 2.1% | 2.1% |
| DABCO | 0.1% | 0.1% | 0.1% | 0.8% | 0.8% | 0.8% |
| Reaction Condition | | | | | | |
| Temperature (° C.) | RT² | RT² | RT² | 60° C. | 60° C. | RT² |
| Reaction Time (minutes) | 10 | 30 | 60 | 10 | 60 | 10 |

¹Percentages are based on total weight of the reaction mixture.
²RT, room temperature.

TABLE 8c

| | Prepolymer (%)¹ | | | | |
|---|---|---|---|---|---|
| | 10 | 11 | 12 | 13 | 14 |
| Reaction Mixture | | | | | |
| Gelatin | 12.4% | 12.4% | 12.4% | 12.4% | 12.4% |
| Benzyl Benzoate | 84.7% | 84.7% | 84.7% | 84.7% | 84.7% |
| TAKENATE ® D-110N | 2.1% | 2.1% | 2.1% | 2.1% | 2.1% |
| DABCO | 0.8% | 0.0% | 0.0% | 0.0% | 0.0% |
| Reaction Condition | | | | | |
| Temperature (° C.) | RT² | 60° C. | 60° C. | RT² | RT² |
| Reaction Time (minutes) | 60 | 10 | 60 | 10 | 60 |

¹Percentages are based on total weight of the reaction mixture.
²RT, room temperature.

TABLE 8d

| Prepolymer | Polyisocyanate Conversion |
|---|---|
| 1 | 35% |
| 2 | 58% |
| 3 | 66% |
| 1A | 0% |
| 2A | 0% |
| 3A | 16% |
| 4 | 10% |
| 5 | 19% |
| 6 | 27% |
| 7 | 86% |
| 8 | 100% |
| 9 | 28% |
| 10 | 60% |
| 11 | 0% |
| 12 | 17% |
| 13 | 0% |
| 14 | 1% |

Based upon the results presented in Tables 7-8d, EE and polyisocyanate conversion are influenced by reaction time, reaction temperature and/or catalyst concentrations during the preparation of prepolymer. DABCO is important to the formation of the prepolymer. Furthermore, the fact that the polyisocyanate conversion is low in the absence gelatin validates the formation of prepolymer.

### Example 3: Gelatin Comparative Microcapsule 15

In this example, the microcapsule slurry was prepared as described in Example 1 (Microcapsule 1), except that the oil phase was prepared without gelatin, and separately, an aqueous solution containing gum Arabic and gelatin was prepared at 60° C. and subsequently emulsified with the oil phase at 6000 rpm for 3 minutes to form an emulsion. The prepared microcapsule slurry was designated Comparative Microcapsule 15.

### Example 4: Gelatin Comparative Microcapsule 16

In this example, the microcapsule slurry was prepared as described in Example 1 (Microcapsule 1), except that the oil phase was prepared without gelatin and polyisocyanate, polyisocyanate was not used in this example, and separately, an aqueous solution containing gum Arabic and gelatin was prepared at 60° C. and subsequently emulsified with the oil phase at 6000 rpm for 3 minutes to form an emulsion. The prepared microcapsule slurry was designated Comparative Microcapsule 16.

### Example 5: Microcapsule pH Stability

The pH of the microcapsule slurries prepared in Example 1 (Microcapsule 1) and Example 3 (Comparative Microcapsule 15) were adjusted to pH 3.5, 5 and greater than 10 respectively using 10% NaOH and the microcapsule stability was monitored using a Motic BA310 microscope. The results of the microscope study indicated that the microcapsule wall of Microcapsule 1 was stable at pH 3.5, 5 and >10. In contrast, the microcapsule wall of Comparative Microcapsule 15 dissolved at pH 5 and >10.

### Example 6: Validation of Prepolymer Formation

In this example, the process of Example 2 to make Prepolymer 1 was repeated. At the end of the prepolymer preparation of Prepolymer 1, a 50 mg aliquot of the oil phase reaction mixture was taken and dissolved in dimethyl sulfoxide/lithium chloride solution and analyzed by multi-angle light scattering coupled with size exclusion chromatography (SEC-MALS) to determine the molar mass. For comparison, gelatin (same as used for the preparation of Prepolymer 1) was also dissolved and analyzed under the same condition. The results of this analysis (Table 9) indicate that Prepolymer 1 had higher molecular weights and larger size comparing with gelatin, which demonstrated the formation of the isocyanate-functionalized prepolymer (gelatin covalently bonded with polyisocyanate).

TABLE 9

| Sample | Mw (kDa)¹ | Mz (kDa)² | PD (Mw/Mn)³ | rz (nm)⁴ |
|---|---|---|---|---|
| Prepolymer | 359 | 1520 | 6.3 | 24 |
| Gelatin | 171 | 401 | 2.2 | 14 |

¹Mw, weight average molecular weight.
²Mz, z-average molecular weight.
³PD, polydispersity. Mn is number average molecular weight.
⁴rz, z-average of root mean square radius.

### Example 7: Validation of Highly Crosslinked Microcapsule Wall

Microcapsules prepared according to Example 1 (Microcapsule 1), Example 3 (Comparative Microcapsule 15), and Example 4 (Comparative Microcapsule 16) were respectively washed 3 times with water to remove any residual unreacted materials. The microcapsules were then freeze dried to remove excess water and then extracted with methanol several times until standard gas chromatography (GC) analysis indicated that the residual fragrance in the microcapsule is less than 2%. Microcapsule samples were then dried and analyzed (at the same weight percentage of dried material) using SEC-MALS to determine the molar mass and the degree of crosslinking. The results of this analysis (Table 10) demonstrated that the microcapsule walls in Microcapsule 1 have significantly higher molecular weight (Mw, Mz) and significantly larger size (rz) compared to the molecular weight and size of the microcapsule walls in Comparative Microcapsules 15 and 16.

PD refers to “polydispersity” and characterizes the distribution of the molecular weights for a given polymer sample. PD is defined as M_{w}/Mₙ which is the weight average molecular weight (M_{w}) divided by the number average molecular weight (Mₙ) of the polymer. When the PD is 1, the polymers in the sample are monodisperse (i.e., all polymers have consistent chain length and molecular weight). PD values greater than 1 mean that the polymers in the sample are polydisperse (i.e., the polymers have non-uniform chain length and molecular weight). The greater the PD value the more polydisperse are the polymers in the sample. The results of this analysis (Table 10) demonstrated that the microcapsule walls in Microcapsule 1 have significantly higher molecular weight polymers and significantly higher polydispersity, indicating higher degree of crosslinking compared with the microcapsule walls in Comparative Microcapsules 15 and 16.

TABLE 10

| | Mw (kDa)¹ | Mz (kDa)² | PD (M_{w}/Mₙ)³ | rz (nm)⁴ |
|---|---|---|---|---|
| Microcapsule 1 | 1218 | 6551 | 3.929 | 38.1 |
| Comparative | 435 | 1075 | 2.332 | 15 |
| Microcapsule 15 | | | | |
| Comparative | 419 | 1104 | 2.273 | 17 |
| Microcapsule 16 | | | | |

¹Mw, weight average molecular weight.
²Mz, z-average molecular weight.
³PD, polydispersity. Mn is number average molecular weight.
⁴rz, z-average of root mean square radius.

Light scattering chromatography is a measurement of concentration and mass of the particles. Gum Arabic and gelatin were eluted at between 66 minutes and 86 minutes and showed up as signature peaks. Notably, microcapsule walls prepared according to Example 3 (Comparative Microcapsule 15) and Example 4 (Comparative Microcapsule 16) had minimal differences indicative of almost no crosslinking or increase in molar mass of either the gum Arabic or gelatin. By comparison, the microcapsule wall prepared according to Example 1 (Microcapsule 1) showed a significant increase in peak intensity for both gum Arabic and gelatin, indicating the significant increase in molar mass of both gum Arabic and gelatin. Furthermore, peak shifts to the left for both gum Arabic and gelatin, also indicating the increase in molar mass as reflected in Table 10. The resulting increase in molar mass and size of the microcapsule walls in Microcapsule 1 is reflective of a higher degree of crosslinking of the gelatin prepolymer and gum Arabic.

### Example 8: Biodegradation of Microcapsules

Microcapsules prepared according to Example 1 (Microcapsule 1) and Examples 3 and 4 (Comparative Microcapsules 15 and 16) were washed, freeze dried and extracted as described above in Example 7 and evaluated by OECD301F. The microcapsule in Microcapsule 1, a capsule according to the present disclosure, was determined to have a biodegradation rate of 75% within 60 days. The biodegradation rate for the microcapsules in Comparative Microcapsules 15 and 16 (not within the scope of the present disclosure) were more than 60% within 60 days. It was also observed that the microcapsule in Microcapsule 1 did not contain a blend of biodegradable and non-biodegradable materials by analysis of capsule wall. In contrast, the microcapsule in Comparative Microcapsule 15 was observed to contain a blend of biodegradable and non-biodegradable materials. The microcapsule in Comparative Microcapsule 16, which is just a gelatin coacervate without polyisocyanate, while it may be biodegradable, was observed to lack performance, stability and/or chemical cross-linking.

### Example 9: Gelatin Prepolymer with Alginate Microcapsules 17-24

An oil phase was prepared by mixing Magnitude fragrance (amount indicated in Table 11), caprylic/capric triglyceride (amount indicated in Table 11, NEOBEE® M-5) and polyisocyanate (amount indicated in Table 11, Takenate® D-110N). Gelatin (amount indicated in Table 11, commercially available from Gelita USA Inc., Sergeant Bluff, IA) was subsequently dispersed in the oil phase under constant mixing. The oil phase mixture was heated with constant mixing and 1,4-diazabicyclo[2.2.2]octane (DABCO, amount indicated in Table 11) was added and heated at 60° C. for 10 min. Separately, an aqueous solution of Gelatin (amount indicated in Table 11) was prepared and emulsified with the oil phase at 6000 rpm for 3 min to form an emulsion. An aqueous solution of Alginate (amount indicated in Table 11, commercially available from International Flavors & Fragrances Inc., NY) was then added to the emulsion as it was kept agitated (300 RPM) at 40° C. After allowing the emulsion to stir at 40° C. for 1 hour, the pH was gradually adjusted to 4.4 using 50% Citric Acid (amount indicated in Table 11). A first aqueous crosslinking solution containing mixture of sucrose (amount indicated in Table 11) and sodium periodate (amount indicated in Table 11) was prepared and stirred at room temperature for 30 min (minutes). The first aqueous crosslinking solution was then added to the emulsion. Stirring of the emulsion was maintained at 40° C. for 4 hrs (hours) for curing and the emulsion was cooled back to room temperature to provide Microcapsule 17.

Separately, Microcapsule 18 was made with the same process as for Microcapsule 17, except that the microcapsule was prepared with further process step of pH adjustment, that is, after the curing, the pH of the microcapsule slurry was adjusted down to pH 3 with concentrated HCl or 50% citric acid to provide Microcapsule 18.

Separately, Microcapsule 19 was made with the same process as for Microcapsule 18, except that after the curing and after the pH of the microcapsule slurry was adjusted down to pH 3, an aqueous calcium nitrate solution (amount indicated in Table 11) was added to the emulsion as a second crosslinking solution, and the resulting emulsion was stirred for an additional 30 min at room temperature to provide Microcapsule 19.

Separately, Microcapsule 20 was made with the same process as for Microcapsule 19, except that when an aqueous solution of Gelatin was prepared and emulsified with the oil phase at 6000 rpm for 3 min to form an emulsion, the aqueous solution also comprises gum Arabic (amount indicated in Table 11).

Separately, Microcapsule 21 was made with the same process as for Microcapsule 19, except that sodium polystyrene sulfonate (amount indicated in Table 11, commercially available under the tradename Flexan® II, Nouryon, The Netherlands) was added as an emulsifier together with the aqueous solution of Gelatin to be emulsified with the oil phase.

Separately, Microcapsule 22 was made with the same process as for Microcapsule 19, except that transglutaminase (amount indicated in Table 11, commercially available from International Flavors & Fragrances Inc., NY) was used in replace of the first aqueous crosslinking solution.

Separately, Microcapsule 23 was made with the same process as for Microcapsule 19, except that the first aqueous crosslinking solution was added after allowing the emulsion to stir at 40° C. for 1 hour but before the pH was gradually adjusted to 4.4 using 50% Citric Acid.

Separately, Microcapsule 24 was made with the same process as for Microcapsule 19, except that the temperature was raised to the range of 65° C. to 75° C. prior to the addition of the first aqueous crosslinking solution.

Separately, Microcapsule 62 was made with the same process as for Microcapsule 19, except that after the pH was gradually adjusted to 4.4 using 50% Citric Acid, the emulsion was cured at 40° C. for 3 hrs, then transglutaminase (amount indicated in Table 11) was used in replace of the first aqueous crosslinking solution, followed with further curing at 40° C. for an additional hour, then the emulsion was cooled to room temperature and tannic acid (amount indicated in Table 11) was added to the emulsion as one of the second crosslinkers, and then the pH of the emulsion was adjusted to 3.5 before the addition of the aqueous calcium nitrate solution.

Separately, Microcapsule 63 was made with the same process as for Microcapsule 62, except that transglutaminase was not used (the first aqueous crosslinking solution was not used either).

TABLE 11

| Microcapsule | 17 | 18 | 19 | 20 | 21 | 22 | 23 | 24 | 62 | 63 |
|---|---|---|---|---|---|---|---|---|---|---|
| Water | 72.8% | 72.72% | 72.5% | 72.5% | 72.5% | 73.35% | 72.5% | 72.5% | 72.60% | 73.35% |
| Gelatin (in | 0.75% | 0.75% | 0.75% | 0.75% | 0.75% | 0.75% | 0.75% | 0.75% | 0.75% | 0.75% |
| aqueous phase) | | | | | | | | | | |
| Gelatin (in oil | 1.5% | 1.5% | 1.5% | 1.5% | 1.5% | 1.5% | 1.5% | 1.5% | 1.50% | 1.50% |
| phase) | | | | | | | | | | |
| Magnitude | 18% | 18% | 17% | 17% | 17% | 17% | 17% | 17% | 17% | 17% |
| Fragrance | | | | | | | | | | |
| Neobee M-5 | 4.5% | 4.5% | 4.25% | 4.25% | 4.25% | 4.25% | 4.25% | 4.25% | 4.25% | 4.25% |
| Takenate D-110N | 0.25% | 0.25% | 0.25% | 0.25% | 0.25% | 0.38% | 0.25% | 0.25% | 0.38% | 0.25% |
| Citric Acid | 0.34% | 0.33% | 0.13% | 0.13% | 0.13% | 0.13% | 0.13% | 0.13% | 0.13% | 0.13% |
| Alginate | 0.5% | 0.5% | 0.5% | 0.5% | 0.5% | 0.5% | 0.5% | 0.5% | 0.5% | 0.5% |
| Sucrose | 1.0% | 1.0% | 1.0% | 1.0% | 1.0% | none | 1.0% | 1.0% | none | none |
| Sodium | 0.10% | 0.10% | 0.10% | 0.10% | 0.10% | none | 0.10% | 0.10% | none | none |
| Periodate | | | | | | | | | | |
| DABCO | 0.015% | 0.015% | 0.015% | 0.015% | 0.015% | 0.015% | 0.015% | 0.015% | 0.015% | 0.015% |
| HCl | none | 0.08% | 0.08% | 0.08% | 0.08% | 0.08% | 0.08% | 0.08% | 0.08% | 0.08% |
| (adjust pH to 3) | | | | | | | | | | |
| Calcium Nitrate | none | none | 1.67% | 1.67% | 1.67% | 1.67% | 1.67% | 1.67% | 1.67% | 1.67% |
| tetrahydrate | | | | | | | | | | |
| Gum Arabic | none | none | none | 0.25% | none | none | none | none | none | none |
| Flexan ® II | none | none | none | none | 0.25% | none | none | none | none | none |
| Tgase | none | none | none | none | none | 0.25% | none | none | 0.5% | none |
| Tannic Acid | none | none | none | none | none | none | none | none | 0.25% | 0.25% |
| EE | 99.4% | 99.4% | 99.4% | 99.4% | 99% | 99.4% | 99.4% | 99.4% | 99.4% | 99.4% |

Note:
(1) Percentages (except EE) are based on total weight of the microcapsule slurry (i.e., emulsion).
(2) Tgase is transglutaminase.

### Example 10: Gelatin Prepolymer with Glutaraldehyde Microcapsule 25

An oil phase was prepared by mixing Magnitude fragrance (18%), caprylic/capric triglyceride (4.5%, NEOBEE® M-5) and polyisocyanate (0.25%, TAKENATE® D-110N). Gelatin (1.5%, commercially available from Gelita USA Inc., Sergeant Bluff, IA) was subsequently dispersed in the oil phase under constant mixing. The oil phase mixture was heated with constant mixing and 1,4-diazabicyclo[2.2.2]octane (0.015%, DABCO) was added to catalyze the prepolymer formation for the reaction time and temperature indicated in Table 3 for Microcapsule 1. Separately, an aqueous solution of gum Arabic (1.5%) was prepared and then emulsified with the oil phase at 6000 rpm for 3 minutes to form an emulsion. After allowing the emulsion to stir at 40° C. for 1 hour, the pH of the emulsion was gradually adjusted to 3.5 using citric acid. A first crosslinking solution containing mixture of sucrose and sodium periodate was prepared and stirred at room temperature for 30 minutes. The first crosslinking solution was then added to the emulsion followed by glutaraldehyde (0.025%, second crosslinker, commercially available from Sigma) and the resulting emulsion was stirred at 40° C. for 4 hours. The microcapsule slurry was then cooled and stirred at room temperature overnight to provide Microcapsule 25. Component percentages in parentheses in Example 10 are based on total weight of the microcapsule slurry (i.e., Microcapsule 25). Citric acid used in Example 10 is a 50% aqueous solution.

### Example 11: Chitosan Prepolymer Microcapsule 26

An oil phase was prepared by mixing Magnitude fragrance (10.0%), caprylic/capric triglyceride (2.5%, NEOBEE® M-5) and polyisocyanate (0.25%, TAKENATE® D-110N). Chitosan (1.5%, available from Glentham Life Sciences, Corsham, UK) was dispersed in the oil phase under constant mixing. The oil phase mixture was heated to 60° C. at constant mixing and DABCO (0.015%) was added to catalyze the reaction for 10 mins. Separately, an aqueous solution of gum Arabic (2.0%) was prepared together with acetic acid (2.0%). The aqueous solution and the oil phase were mixed and emulsified at 6000 rpm for 3 minutes to form an emulsion. The emulsion was stirred at 40° C. for 1 hour, then the pH of the emulsion was gradually adjusted to 5.5 using 5% sodium hydroxide solution. A first aqueous crosslinking solution composed of sucrose (1.0%) and sodium periodate (0.1%) was prepared and stirred at room temperature for 30 minutes. The first aqueous crosslinking solution was then added to the emulsion and the stirring was maintained at 40° C. for 4 hours to provide the microcapsule slurry (i.e., Microcapsule 26). Component percentages in parentheses in Example 11 are based on total weight of the microcapsule slurry (i.e., Microcapsule 26).

### Example 12: Chitosan Prepolymer with Tannic Acid Microcapsules 27-30

An oil phase was prepared by mixing Magnitude fragrance (18.0%), caprylic/capric triglyceride (4.5%, NEOBEE® M-5) and polyisocyanate (0.50%, TAKENATE® D-110N). Chitosan (amount indicated in Table 12, available from KitoZyme, Herstal, Belgium) was dispersed in the oil phase under constant mixing. The oil phase mixture was heated to 60° C. at constant mixing and DABCO (0.015%) was added to catalyze the reaction for 10 min. Separately, an aqueous solution containing gum Arabic (amount indicated in Table 12) and acetic acid (1.0%) was prepared. The aqueous solution and the oil phase were mixed and emulsified at 6000 rpm for 3 minutes to form an emulsion. The emulsion was stirred at 40° C. for 1 hour, then the pH of the emulsion was gradually adjusted to 5.0 using 5% sodium hydroxide solution. A first aqueous crosslinking solution composed of sucrose (1.0%) and sodium periodate (0.1%) was prepared and stirred at room temperature for 30 minutes. The first aqueous crosslinking solution was then added to the emulsion followed by the addition of tannic acid (second crosslinker, amount indicated in Table 12, sold under the tradename Tanal 02®) to the emulsion and the resulting emulsion maintained stirring at 40° C. for 4 hours (i.e., curing) to provide the microcapsule slurry (i.e., Microcapsules 27-30 respectively). The microcapsule slurry was then cooled and stirred at room temperature overnight. Component percentages in parentheses in Example 12 are based on total weight of the microcapsule slurry.

TABLE 12

| Microcapsule | 27 | 28 | 29 | 30 |
|---|---|---|---|---|
| Water | 67.9% | 67.9% | 67.9% | 67.9% |
| Gum Arabic | 3.0% | 3.0% | 4.0% | 4.0% |
| Chitosan | 1.5% | 1.5% | 2.0% | 2.0% |
| Magnitude Fragrance | 18% | 18% | 18% | 18% |
| Neobee M-5 | 4.5% | 4.5% | 4.5% | 4.5% |
| Takenate D-110N | 0.50% | 0.50% | 0.50% | 0.50% |
| Acetic Acid | 1.0% | 1.0% | 1.0% | 1.0% |
| NaOH | 2.0% | 2.0% | 2.0% | 2.0% |
| Sucrose | 1.0% | 1.0% | 1.0% | 1.0% |
| Sodium Periodate | 0.10% | 0.10% | 0.10% | 0.10% |
| DABCO | 0.015% | 0.015% | 0.015% | 0.015% |
| Tannic Acid | 0.25% | 1.00% | 0.25% | 1.00% |
| EE | 99% | 99% | 99% | 99% |

Note:
(1) EE means encapsulation efficiency;
(2) percentages (except EE) are based on total weight of the microcapsule slurry (i.e., emulsion).

### Example 13: Chitosan Prepolymer with Other Crosslinker Microcapsules 31-36

Microcapsules 31-36 were prepared following the process of Example 12 except with different second crosslinkers and certain different component amounts as shown in Table 13.

TABLE 13

| Microcapsule | 31 | 32 | 33 | 34 | 35 | 36 |
|---|---|---|---|---|---|---|
| Water | 67.9% | 67.9% | 67.9% | 67.9% | 67.9% | 67.9% |
| Gum Arabic | 3.0% | 3.0% | 3.0% | 3.0% | 3.0% | 4.0% |
| Chitosan | 1.5% | 1.5% | 1.5% | 1.5% | 1.5% | 2.0% |
| Magnitude | 18% | 18% | 18% | 18% | 18% | 18% |
| Fragrance | | | | | | |
| Neobee M-5 | 4.5% | 4.5% | 4.5% | 4.5% | 4.5% | 4.5% |
| Takenate D-110N | 0.50% | 0.50% | 0.50% | 0.50% | 0.50% | 0.50% |
| Acetic Acid | 1.0% | 1.0% | 1.0% | 1.0% | 1.0% | 1.0% |
| NaOH | 2.0% | 2.0% | 2.0% | 2.0% | 2.0% | 2.0% |
| Sucrose | 1.0% | 1.0% | 1.0% | 1.0% | 1.0% | 1.0% |
| Sodium Periodate | 0.10% | 0.10% | 0.10% | 0.10% | 0.10% | 0.10% |
| DABCO | 0.015% | 0.015% | 0.015% | 0.015% | 0.015% | 0.015% |
| Second Crosslinker | Glutaraldehyde | Glutaraldehyde | Glyoxal | Picassian ® | Picassian ® | TEOS |
| | | | | XL-702 | XL-732 | |
| | 0.25% | 1.00% | 0.25% | 0.25% | 0.25% | 1.00% |
| EE | 99% | 48% | 99% | 99% | 99% | 85% |

Notes:
(1) Picassian ® XL-702 is a polycarbodiimide;
(2) EE means encapsulation efficiency;
(3) percentages (except EE) are based on total weight of the microcapsule slurry (i.e., emulsion);
(4) TEOS is tetraethoxysilane.

### Example 14: Chitosan Prepolymer Without Catalyst Microcapsule 37

An oil phase was prepared by mixing Magnitude fragrance (18.0%), caprylic/capric triglyceride (4.5%, NEOBEE® M-5) and polyisocyanate (0.50%, TAKENATE® D-110N). Chitosan (2.0%, available from KitoZyme, Herstal, Belgium) was dispersed in the oil phase under constant mixing. The oil phase mixture was heated to 60° C. with constant mixing for 20 min. Separately, an aqueous solution containing gum Arabic (4.0%) and acetic acid (1.0%) was prepared. The aqueous solution and the oil phase were mixed and emulsified at 6000 rpm for 3 minutes to form an emulsion. The emulsion was stirred at 40° C. for 1 hour, then the pH of the emulsion was gradually adjusted to 5.0 using 5% sodium hydroxide solution. A first aqueous crosslinking solution composed of sucrose (1.0%) and sodium periodate (0.1%) was prepared and stirred at room temperature for 30 minutes. The first aqueous crosslinking solution was then added to the emulsion followed by addition of tannic acid (second crosslinker, 0.25%, sold under the tradename Tanal 02®) to the emulsion and the resulting emulsion maintained stirring at 40° C. for 4 hours to provide the microcapsule slurry (i.e., Microcapsule 37). The microcapsule slurry was then cooled and stirred at room temperature overnight. Component percentages in parentheses in Example 14 are based on total weight of the microcapsule slurry.

### Example 15: Chitosan Prepolymer with Pea Protein Co-Emulsifier Microcapsule 38

An oil phase was prepared by mixing Magnitude fragrance (18.0%), caprylic/capric triglyceride (4.5%, NEOBEE® M-5) and polyisocyanate (0.50%, TAKENATE® D-110N). Chitosan (2.0%, available from KitoZyme, Herstal, Belgium) was dispersed in the oil phase under constant mixing. The oil phase mixture was heated to 60° C. at constant mixing and DABCO (0.015%) was added to catalyze the reaction for 10 min. Separately, an aqueous solution containing gum Arabic (4.0%), pea protein (1%, commercially available from International Flavors & Fragrances Inc., NY) and acetic acid (1.0%) was prepared. The aqueous solution and the oil phase were mixed and emulsified at 6000 rpm for 3 minutes to form an emulsion. The emulsion was stirred at 40° C. for 1 hour, then the pH of the emulsion was gradually adjusted to 5.0 using 5% sodium hydroxide solution. A first aqueous crosslinking solution composed of sucrose (1.0%) and sodium periodate (0.1%) was prepared and stirred at room temperature for 30 minutes. The first aqueous crosslinking solution was then added to the emulsion followed by addition of tannic acid (second crosslinker, 0.25%, sold under the tradename Tanal 02®) to the emulsion and the resulting emulsion maintained stirring at 40° C. for 4 hours to provide the microcapsule slurry (i.e., Microcapsule 38). The microcapsule slurry was then cooled and stirred at room temperature overnight. Component percentages in parentheses in Example 15 are based on total weight of the microcapsule slurry.

### Example 16: Chitosan, Pea Protein Prepolymer Microcapsule 39

An oil phase was prepared by mixing Magnitude fragrance (18.0%), caprylic/capric triglyceride (4.5%, NEOBEE® M-5) and polyisocyanate (0.50%, TAKENATE® D-110N). Chitosan (2.0%, available from KitoZyme, Herstal, Belgium) and pea protein (0.1%, available from International Flavors & Fragrances Inc., NY) were dispersed in the oil phase under constant mixing. The oil phase mixture was heated to 60° C. at constant mixing and DABCO (0.015%) was added to catalyze the reaction for 10 min. Separately, an aqueous solution containing gum Arabic (4.0%), pea protein (0.9%) and acetic acid (1.0%) was prepared. The aqueous solution and the oil phase were mixed and emulsified at 6000 rpm for 3 minutes to form an emulsion. The emulsion was stirred at 40° C. for 1 hour, then the pH of the emulsion was gradually adjusted to 5.0 using 5% sodium hydroxide solution. A first aqueous crosslinking solution composed of sucrose (1.0%) and sodium periodate (0.1%) was prepared and stirred at room temperature for 30 minutes. The first aqueous crosslinking solution was then added to the emulsion followed by addition of tannic acid (0.25%, sold under the tradename Tanal 02®) to the emulsion and the resulting emulsion maintained stirring at 40° C. for 4 hours to provide the microcapsule slurry (i.e., Microcapsule 39). The microcapsule slurry was then cooled and stirred at room temperature overnight. Component percentages in parentheses in Example 16 are based on total weight of the microcapsule slurry.

### Example 17: Chitosan Prepolymer with Co-Solvent 3-Methoxybutyl Acetate Microcapsule 40

An oil phase was prepared by mixing Magnitude fragrance (9.0%), 3-methoxybutyl acetate (5.0%, available from Sigma-Aldrich, St. Louis, MO), and polyisocyanate (0.50%, TAKENATE® D-110N). Chitosan (2.0%, available from KitoZyme, Herstal, Belgium) was dispersed in the oil phase under constant mixing. The oil phase mixture was heated to 60° C. at constant mixing and DABCO (0.015%) was added to catalyze the reaction for 10 min. Separately, an aqueous solution containing gum Arabic (4.0%) and acetic acid (1.0%) was prepared. The aqueous solution, the oil phase, an additional Magnitude fragrance (9.0%) and caprylic/capric triglyceride (4.5%, NEOBEE® M-5) were mixed and emulsified at 6000 rpm for 3 minutes to form an emulsion. The emulsion was stirred at 40° C. for 1 hour, then the pH of the emulsion was gradually adjusted to 5.0 using 5% sodium hydroxide solution. A first aqueous crosslinking solution composed of sucrose (1.0%) and sodium periodate (0.1%) was prepared and stirred at room temperature for 30 minutes. The first aqueous crosslinking solution was then added to the emulsion followed by addition of tannic acid (0.25%, sold under the tradename Tanal 02®) to the emulsion and the resulting emulsion maintained stirring at 40° C. for 4 hours to provide the microcapsule slurry (i.e., Microcapsule 40). The microcapsule slurry was then cooled and stirred at room temperature overnight. Component percentages in parentheses in Example 17 are based on total weight of the microcapsule slurry.

### Example 18: Chitosan Prepolymer with Co-Solvent Phenyl Ethyl Benzoate Microcapsule 41

An oil phase was prepared by mixing phenyl ethyl benzoate (10.0%, commercially available from International Flavors & Fragrances Inc., NY) and polyisocyanate (0.50%, TAKENATE® D-110N). Chitosan (2.0%, available from KitoZyme, Herstal, Belgium) was dispersed in the oil phase under constant mixing. The oil phase mixture was heated to 60° C. at constant mixing and DABCO (0.015%) was added to catalyze the reaction for 10 min. Separately, an aqueous solution containing gum Arabic (4.0%) and acetic acid (1.0%) was prepared. The aqueous solution, the oil phase, a Magnitude fragrance (18.0%), and caprylic/capric triglyceride (4.5%, NEOBEE® M-5) were mixed and emulsified at 6000 rpm for 3 minutes to form an emulsion. The emulsion was stirred at 40° C. for 1 hour, then the pH of the emulsion was gradually adjusted to 5.0 using 5% sodium hydroxide solution. A first aqueous crosslinking solution composed of sucrose (1.0%) and sodium periodate (0.1%) was prepared and stirred at room temperature for 30 minutes. The first aqueous crosslinking solution was then added to the emulsion followed by addition of tannic acid (0.25%, sold under the tradename Tanal 02®) to the emulsion and the resulting emulsion maintained stirring at 40° C. for 4 hours to provide the microcapsule slurry (i.e., Microcapsule 41). The microcapsule slurry was then cooled and stirred at room temperature overnight. Component percentages in parentheses in Example 18 are based on total weight of the microcapsule slurry.

### Example 19: Prepolymer Microcapsules 42-49 with Various Biopolymers and Polyelectrolytes

Component percentages in parentheses in Example 19 are based on total weight of the microcapsule slurry. Microcapsule 42 was prepared following Example 1 except that Gelatin (1.5%) was replaced with Cationic guar (1.0%, available as N-HANCE™ C261N from Ashland Specialty Chemical, Delaware), and after allowing the emulsion to stir at 40° C. for 1 hour, the pH of the emulsion was gradually adjusted to 3.8 using 50% citric acid.

Microcapsule 43 was prepared following Example 1 except that Gelatin (1.5%) was replaced with Cationic glucan (1.5%, having a degree of substitution per monomer glucan unit of less than 10%), and after allowing the emulsion to stir at 40° C. for 1 hour, the pH of the emulsion was gradually adjusted to 2.8 using 50% citric acid.

Microcapsule 44 was prepared following Example 1 except that Gelatin (1.5%) was replaced with Collagen (10.0%, Biollagen™ available from JLand Biotech Co. Ltd., Jinjiang, China), gum Arabic (2.0%) was replaced with gum Arabic (3.0%), polyisocyanate (0.25%, TAKENATE® D-110N) was replaced with polyisocyanate (0.50%, TAKENATE® D-110N), and after allowing the emulsion to stir at 40° C. for 1 hour, the pH of the emulsion was gradually adjusted to 2.75 using 50% citric acid.

Microcapsule 45 was prepared following Example 1 except that Gelatin (1.5%) was replaced with gum Arabic (2.0%), and gum Arabic (2.0%) was replaced with Gelatin (1.5%).

Microcapsule 46 was prepared following Example 1 except that Gelatin (1.5%) was replaced with Pea Protein (1.5%, commercially available as Nutralys® S85XF from Roquette, France), and after allowing the emulsion to stir at 40° C. for 1 hour, the pH of the emulsion was gradually adjusted to 2.0 using 50% citric acid.

Microcapsule 47 was prepared following Example 1 except that gum Arabic (2.0%) was replaced with carboxymethyl cellulose (2.0%, prepared according to EP 2552968 B1 with Degree of Substitution (DS) of 0.4), and after allowing the emulsion to stir at 40° C. for 1 hour, the pH of the emulsion was gradually adjusted to 4.6 using 50% citric acid.

Microcapsule 48 was prepared following Example 1 except that gum Arabic (2.0%) was replaced with carboxymethyl cellulose (2.0%, prepared according to EP 2552968 B1 with Degree of Substitution (DS) of 0.2), and after allowing the emulsion to stir at 40° C. for 1 hour, the pH of the emulsion was gradually adjusted to 4.6 using 50% citric acid.

Microcapsule 49 was prepared following Example 1 except that gum Arabic (2.0%) was replaced with pectin (2.0%, commercially available as GENU® Pectin from CP Kelco, GA).

### Example 20. Encapsulation Efficiencies (EE) of Microcapsules 42-49

The encapsulation efficiencies of Microcapsules 42-49 were analyzed and are presented in Table 14.

TABLE 14

| Microcapsule | EE |
|---|---|
| 42 | 99% |
| 43 | 99% |
| 44 | 97% |
| 45 | 99% |
| 46 | 75% |
| 47 | 98% |
| 48 | 99% |
| 49 | 85% |

The above EE results demonstrated that isocyanate-functionalized prepolymers can be formed with biopolymers having amine (e.g., —NH₂), hydroxyl (—OH) and/or carboxyl (—COOH) functional groups, such as gelatin (—NH₂), chitosan (—NH₂, —OH), modified guar (—OH), modified glucans (—OH), collagen (—NH₂, —OH), proteins (—NH₂, —OH), gum Arabic (—OH, —COOH), alginate (—OH, —COOH) and/or pectin (—OH, —COOH). Such isocyanate-functionalized prepolymers can be used in microencapsulation according to the present disclosure.

### Example 21: Microcapsule Performance

To evaluate fragrance profile performance, a testing microcapsule slurry was blended into a model fabric conditioner base (19% active level) as shown in Table 15. The testing microcapsule slurry was same as Microcapsule 1 except its microcapsules encapsulated two different fragrances, Magnitude fragrance and Mermaid fragrance.

TABLE 15

| Material | Amount (g) |
|---|---|
| REWOQUAT ® WE 18 | 23.46 |
| (Esterquat; Evonik, 90% in IPA) | |
| PROXEL ® GLX (1,2- | 0.11 |
| benzisothiazolin-3-one) | |
| Water | 76.1 |
| Calcium Chloride (25%) | 0.33 |

Note:
IPA means isopropyl alcohol.

The fragrance load was 0.1% neat oil equivalent (NOE). The fragrance intensity of the perfumes encapsulated by the testing microcapsule was evaluated by conducting a laundry experiment using accepted experimental protocols using European wash machine (Miele). Terry towels were used for the washing experiments and were washed with the model fabric conditioner containing the testing microcapsule slurry. Washed terry towel samples were removed from the washing machine and line dried overnight. The samples were evaluated by a panel of 24 trained judges at three different stages (pre-rub, gentle handling, and post-rub) and rated on a scale ranging from 0 to 30.

“Pre-rub” refers to the evaluation of the towels by panelists before the folding of the towels. “Gentle handling” refers to the folding of the towels twice, followed by the evaluation of the towels by the panelists. “Post-rub” refers to vigorous application of mechanical force using both hands to rub the towels at least once to rupture the testing microcapsule and then evaluate for signs of released fragrance. A numerical value of 0 indicates that the fabric produced no signs of released fragrance, 5 indicates that the fabric only produced weak intensity and 30 indicates a very strong smell of released fragrance from the testing microcapsules.

The results indicated that the testing microcapsule slurry demonstrated intensity levels of 7.9 at “Pre-rub” and 13.6 at “Post-rub”, respectively, showing superior performance as compared to test results of neat fragrances (i.e., non-encapsulated fragrances) which showed intensity levels below 4 at both “Pre-rub” and “Post-rub” stages.

### Example 22: Encapsulated High Performance Fragrance Compositions

High Performance fragrance Samples 1 and 2 are provided in Table 16 and represent formulations of encapsulated fragrance comprising High Performance fragrance ingredients according to the present disclosure. Comparative fragrance Sample 1 is also provided in Table 16, which represents encapsulated standard fragrance not intended to form the High Performance fragrance of the present disclosure. The fragrance formulations were made by mixing the listed ingredients in the listed proportions in Table 16 at room temperature. The microcapsules used were Example 1 (Microcapsule 1) and Examples 3 and 4 (Comparative Microcapsules 15 and 16).

TABLE 16

| | | | High | High |
|---|---|---|---|---|
| | | Comparative | Performance | Performance |
| Fragrance | | Fragrance | Fragrance | Fragrance |
| Ingredient Name | CAS Number | Sample 1 | Sample 1 | Sample 2 |
| ADOXAL TOCO | 141-13-9 | 14.29 | 35.00 | 0.00 |
| ALD AA TRIPLAL TOCO | 68039-49-6 | 28.57 | 60.00 | 65.00 |
| ALD C-10 TOCO | 112-31-2 | 0.00 | 0.00 | 75.00 |
| ALD C-12 MNA TOCO | 110-41-8 | 0.00 | 0.00 | 120.00 |
| ALLYL AMYL | 67634-00-8 | 28.57 | 0.00 | 0.00 |
| GLYCOLATE BHT | | | | |
| MUSK INDENOFURAN | 476332-65-7, | 8.57 | 25.00 | 6.00 |
| | 647828-16-8 | | | |
| AMBERTONIC | 1392325-86-8 | 0.00 | 0.00 | 15.00 |
| CASHMERAN | 33704-61-9 | 0.00 | 0.00 | 45.00 |
| CYCLACET | 2500-83-6 | 285.71 | 75.00 | 0.00 |
| CYCLAPROP | 68912-13-0 | 142.86 | 184.50 | 0.00 |
| CYCLOHEXYL SAL | 25485-88-5 | 0.00 | 0.00 | 21.00 |
| (ELINCS) | | | | |
| DAMASCONE DELTA | 71048-82-3 | 0.00 | 0.00 | 50.00 |
| TOCO | | | | |
| DIHYDRO TERPINEOL | 498-81-7 | 4.06 | 0.00 | 0.00 |
| DIHYDRO TERPINYL | 58985-18-5 | 94.29 | 0.00 | 0.00 |
| ACET | | | | |
| EUCALYPTOL USP CSM | 470-82-6 | 0.00 | 0.00 | 100.00 |
| FRUCTALATE (ELINCS) | 72903-27-6 | 0.00 | 0.00 | 25.00 |
| GALBANUM OIL LMR ¹ | natural oil | 0.00 | 0.40 | 0.50 |
| GALBASCONE | 56973-85-4 | 0.00 | 10.00 | 4.00 |
| ALPHA 95 PRG TOCO | | | | |
| GALBASCONE | 56973-85-4, | 22.86 | 50.00 | 15.00 |
| PRG TOCO | 56973-84-3 | | | |
| GERANIOL 980 PURE | 106-24-1 | 0.00 | 0.00 | 15.00 |
| HERBALIME | 8006-64-2 | 0.00 | 0.00 | 40.00 |
| HEXYL CINN ALD TBHQ | 101-86-0 | 0.00 | 5.10 | 0.00 |
| ISO BORNYL ACET | 125-12-2 | 182.86 | 80.00 | 50.00 |
| JAVAMOR TT (ELINCS) | 198404-98-7 | 0.00 | 0.00 | 4.00 |
| MANZANATE | 39255-32-8 | 0.00 | 0.00 | 35.00 |
| MELONAL TOCO | 106-72-9 | 0.00 | 0.00 | 30.00 |
| METH DH JASMONATE | 24851-98-7 | 0.00 | 35.00 | 0.00 |
| METH IONONE | 127-51-5 | 0.00 | 0.00 | 150.00 |
| GAMMA “FF” TOCO | | | | |
| METH IONONE | 127-51-5 | 0.00 | 40.00 | 0.00 |
| GAMMA A TOCO | | | | |
| METH PHEN ETH ETHER | 3558-60-9 | 0.00 | 0.00 | 3.00 |
| MONTAVERDI (ELINCS) | 188570-78-7 | 0.00 | 0.00 | 7.50 |
| ORANGE FLOWER | 14576-08-0 | 85.71 | 85.00 | 0.00 |
| ETHER TOCO | | | | |
| PAMPLEZEST | 1465004-85-6 | 0.00 | 0.00 | 15.00 |
| PINO ACETALD TOCO | 33885-51-7 | 0.00 | 0.00 | 40.00 |
| ROSYRANE SUPER | 60335-71-9 | 0.00 | 0.00 | 30.00 |
| TERPINOLENE P UB | 586-62-9 | 57.14 | 130.00 | 0.00 |
| TOCO | | | | |
| TETRAHYDRO | 78-69-3 | 22.76 | 70.00 | 39.00 |
| LINALOOL | | | | |
| TETRAHYDRO | 68480-08-0 | 0.00 | 55.00 | 0.00 |
| LINALYL ACET | | | | |
| TETRAHYDRO | 18479-57-7 | 21.76 | 60.00 | 0.00 |
| MYRCENOL | | | | |
| Total | | 1000 | 1000 | 1000 |

### Example 23: Fragrance Performance with Encapsulated High Performance Fragrances

The fragrance profile performance of the encapsulated fragrance compositions as described in Example 22 was evaluated in a similar way as Example 21. The fabric conditioner (as shown in Table 15) comprising encapsulated fragrance formulations in Table 16 was used for the terry towels in the washing experiments. The terry towels were washed with laundry and were evaluated by 12 trained panelists as described in Example 21. The results from the sensory panelists were then averaged.

The addition of encapsulated fragrance composition comprising High Performance fragrance ingredients of the present disclosure (i.e., High Performance Fragrance Samples 1 & 2) into a fabric conditioner base improved fragrance intensity at pre-rub and gentle handling stages when compared to a benchmark encapsulated fragrance composition (i.e. Comparative Fragrance Sample 1). This Example demonstrated that when using an encapsulated High Performance fragrance according to the present disclosure, fragrance intensity perception at pre-rub and gentle handling stages can be further improved.

### Example 24: Exemplary Consumer Product Compositions

Example 24 demonstrated various consumer products comprising the biodegradable core-shell microcapsule of the present disclosure. Composition A shown in Table 17 is an example of fine fragrance composition according to the present disclosure. It can be prepared by admixture of the components described in Table 17, in the proportions indicated.

TABLE 17

| Ingredient | Composition A (wt % ¹) |
|---|---|
| Ethanol SD-40 | 60-80 |
| Microcapsule Slurry ² | 2.5-25 |
| DI-Water | Balance |
| Total | 100 |

¹ wt % relative to the total weight of the composition.
² Encapsulated fragrance amount depends on the fragrance loading to 0.3% NOE.

Composition B shown in Table 18 is an example of fabric conditioner composition according to the present disclosure. It can be prepared by admixture of the components described in Table 18, in the proportions indicated.

TABLE 18

| Ingredient | Composition B (wt % ¹) |
|---|---|
| Fabric conditioner base of Table 15 | 90 |
| Microcapsule Slurry ² | 0.5-3.0 |
| DI-Water | Balance |
| Total | 100 |

¹ wt % relative to the total weight of the composition.
² Encapsulated fragrance amount depends on the fragrance loading to 0.2% NOE.

Composition C shown in Table 19 is an example of liquid detergent composition according to the present disclosure. It can be prepared by admixture of the components described in Table 19, in the proportions indicated.

TABLE 19

| Ingredient | Composition C (wt % ¹) |
|---|---|
| Black bull Liquid laundry detergent | 90 |
| Microcapsule Slurry ² | 0.5-3.0 |
| DI-Water | Balance |
| Total | 100 |

¹ wt % relative to the total weight of the composition.
² Encapsulated fragrance amount depends on the fragrance loading to 0.2% NOE.

Composition D shown in Table 20 is an example of powder detergent composition according to the present disclosure. It can be prepared by admixture of the components described in Table 20, in the proportions indicated.

TABLE 20

| | Composition D |
|---|---|
| Ingredient | (wt % ¹) |
| Linear alkylbenzene sulfonate | 22 |
| C₁₂₋₁₄ dimethylhydroxyethyl ammonium | 0.2 |
| chloride | |
| AE3S ³ | 1 |
| Zeolite A | 1 |
| 1.6R silicate (SiO₂:Na₂O at ratio 1.6:1) | 5 |
| Sodium carbonate | 20 |
| Polyacrylate Mw 4500 | 0.6 |
| Carboxymethyl cellulose | 0.3 |
| STAINZYME ® amylase (20 mg active/g) | 0.2 |
| Protease (SAVINASE ®, 32.89 mg active/g) | 0.1 |
| Lipase-LIPEX ® (18 mg active/g) | 0.07 |
| Fluorescent brightener | 0.06 |
| DTPA (diethylenetriaminepentaacetic acid) | 0.8 |
| MgSO₄ | 1 |
| Sodium percarbonate | 5.2 |
| TAED (tetra acetyl ethylene diamine) | 1.2 |
| Neat Fragrance | 0.5 |
| Microcapsule Slurry ² | 0.5-3.0 |
| Total | 100 |

¹ wt % relative to the total weight of the composition.
² Encapsulated fragrance amount depends on the fragrance loading to 0.2% NOE.
³ AE3S is sodium lauryl ether sulphate (70%) sold under the brand name Tensagex EOC970B (AE3S).

### Example 25: Evaluation of Chitosan Prepolymer Microcapsules with Second Crosslinker

Comparative Chitosan Prepolymer Microcapsules. Comparative Chitosan Prepolymer Microcapsule 1 was prepared following Example 12 except that the second crosslinker (tannic acid) was not added/used. Comparative Chitosan Prepolymer Microcapsule 2 was prepared following Example 12 except that neither the first crosslinker (oxidized sucrose) nor the second crosslinker (tannic acid) was added/used. Comparative Chitosan Prepolymer Microcapsule 3 was prepared following Example 12 except that glutaraldehyde (instead of the oxidized sucrose) was used as the first crosslinker.

Encapsulation Efficiency (EE). The EE of Microcapsules 27-35 (prepared in Examples 12 and 13) and Comparative Chitosan Prepolymer Microcapsules 1-3 were analyzed and are presented in Table 21.

TABLE 21

| Microcapsule | EE | Free Oil |
|---|---|---|
| 27 | >99% | <0.1% |
| 28 | >99% | <0.1% |
| 29 | >99% | <0.1% |
| 30 | 99% | 0.1% |
| 31 | 99% | 0.1% |
| 32 | 48% | 9.3% |
| 33 | 99% | 0.1% |
| 34 | N/A | N/A |
| 35 | N/A | N/A |
| Comparative 1 | 96% | 0.7% |
| Comparative 2 | 99% | 0.1% |
| Comparative 3 | 67% | 5.9% |

Microcapsule pH Stability. The pH of the microcapsule slurries prepared in Examples 12 and 13 as well as the Comparative Chitosan Prepolymer Microcapsules 1-3 prepared in this Example were adjusted to the values indicated in Table 22 using 50% citric acid or 10% sodium hydroxide. The microcapsule coacervation stability was monitored using a Motic BA310 microscope. The results of this analysis are summarized in Table 22.

TABLE 22

| Microcapsule | pH 3 | pH 5 | pH 10 |
|---|---|---|---|
| 27 | Stable | Stable | Stable |
| 28 | Stable | Stable | Stable |
| 29 | Stable | Stable | Stable |
| 30 | Stable | Stable | Stable |
| 31 | Stable | Stable | Stable |
| 32 | Stable | Stable | Stable |
| 33 | Stable | Stable | Stable |
| Comparative 1 | Not Stable | Stable | Stable |
| Comparative 2 | Not Stable | Stable | Semi-Stable |
| Comparative 3 | Not Stable | Not Stable | Not Stable |

Microcapsule Elevated Temperature Stability. Microcapsules 29, 31, 34 and 35 as well as the Comparative Chitosan Prepolymer Microcapsule 1 were prepared in the same way as the corresponding original Microcapsules described in Examples 12 and 13 and this Example, except that the curing was carried out at 70° C. instead of 40° C. The pH of the microcapsule slurries was then adjusted using 10% sodium hydroxide. The microcapsule coacervation stability under the conditions indicated in Table 23 was monitored using a Moptic BA310 microscope. The results of this analysis are summarized in Table 23.

TABLE 23

| | pH 5 | | pH 10 | |
|---|---|---|---|---|
| | room | pH 5 | room | pH 10 |
| Microcapsule | temperature | 70° C. | temperature | 70° C. |
| 29 | Stable | Stable | Stable | Stable |
| 31 | Stable | Stable | Stable | Stable |
| 34 | Stable | Stable | Stable | Stable |
| 35 | Stable | Stable | Stable | Stable |
| Comparative 1 | Stable | Not Stable | Stable | Not Stable |

Biodegradation of Microcapsules. Microcapsule 29 (Example 12) and Comparative Chitosan Prepolymer Microcapsule 1 (Comparative 1) were washed as described in Example 7 and their biodegradation were evaluated according to OECD301F. Microcapsule 29, a microcapsule according to the present disclosure, was determined to have a biodegradation rate of >60% within 60 days. The biodegradation rate of Comparative 1 was also >60% within 60 days. It was observed that the second crosslinker was able to improve the microcapsule wall stability and the performance of the microcapsule while keeping the biodegradability of the microcapsule wall.

Microcapsule Performance in a Fabric Conditioner. To evaluate fragrance profile performance, a testing microcapsule slurry was blended into the model fabric conditioner base as described in Example 21. The testing microcapsule slurry was same as Microcapsule 29 (Example 12) except its microcapsules encapsulated two different fragrances, Magnitude fragrance and Zazu fragrance and the fragrance load was 0.1% neat oil equivalent (NOE). Using the same evaluation process and scale as described in Example 21, it was observed that the testing microcapsule slurry demonstrated intensity levels of 8.0 at “Pre-rub” and 11.8 at “Post-rub”, which was superior to the performance of neat fragrance (i.e., non-encapsulated fragrance) having “Pre-rub” and “Post-rub” intensities of only 4.

Microcapsule Performance in a Powder Detergent. Microcapsule 29 (Example 12) was added to a powder detergent having the components and proportions indicated in Table 20 except that the fragrance loading was 0.043% NOE. The fragrance intensity of the perfumes encapsulated in the microcapsules of Microcapsule 29 was evaluated by conducting a laundry experiment via accepted experimental protocols using handwashing. Terry towels were used for the washing experiments and were washed with the powder detergent containing Microcapsule 29. Washed terry towel samples were removed and line dried overnight. The samples were evaluated by trained judges at four different stages (soak, damp, pre-rub, and gentle-handling) and rated on a scale ranging from 0 to 5. In accordance with the protocol, “Soak” refers to the evaluation of the towels by the judges when soaking in an aqueous wash containing a dissolved powder detergent product. “Damp” refers to the evaluation of the towels by the judges after the wash while the towels are still wet. “Pre-rub” refers to the evaluation of the towels by the judges before the folding of the towels. “Gentle-handling” refers to the folding of the towels twice to rupture the microcapsules and then evaluation of the towels by the judges for signs of released fragrance. A numerical value of 0 indicates that the fabric produced no signs of released fragrance, whereas 5 indicates that the fabric produced a very strong smell of fragrance released from the microcapsules.

The results indicated that Microcapsule 29 demonstrated intensity levels of 1.8 at “Soak,” 2.6 at “Damp,” 2.3 at Pre-rub and 2.5 at “Gentle-handling.” This was superior to the performance of neat fragrance (i.e., non-encapsulated fragrance), which exhibited an intensity of only 0.5 at all four stages.

### Example 26: Gelatin Prepolymer Microcapsule with Second Crosslinker

An oil phase was prepared by mixing Magnitude fragrance (18%), caprylic/capric triglyceride (4.5%, NEOBEE® M-5) and polyisocyanate (0.25%, TAKENATE® D-110N). Gelatin (1.5%) was subsequently dispersed into the oil phase under constant mixing. The oil phase mixture was heated to 60° C. at constant mixing and DABCO (0.015%) was added to catalyze the reaction for 10 minutes. Separately, an aqueous solution of gum Arabic (2.0%) was prepared and then emulsified with the oil phase at 6000 rpm for 3 minutes to form an emulsion. After allowing the emulsion to stir at 40° C. for 1 hour, the pH of the emulsion was gradually adjusted to 3.5 using 50% citric acid solution. A first aqueous crosslinking solution containing sucrose (1.0%) and sodium periodate (0.1%) was prepared and stirred at room temperature for 30 minutes. The first aqueous crosslinking solution was then added to the emulsion followed by the addition of the second crosslinking solution with the second crosslinker and the amount indicated in Table 24. The resulting emulsion maintained stirring at 40° C. for 4 hours (curing) to provide the microcapsule slurry (i.e., Microcapsules 50-52 respectively). The microcapsule slurry was then cooled and stirred at room temperature overnight. Component percentages in parentheses in Example 26 are based on total weight of the microcapsule slurry.

TABLE 24

| Microcapsule | 50 | 51 | 52 |
|---|---|---|---|
| Second Crosslinker | Tannic Acid | Glutaraldehyde | Glyoxal |
| Amount | 0.25% | 0.25% | 0.25% |

Note:
percentages are based on total weight of the microcapsule slurry (i.e., emulsion).

### Example 27: Cationic Guar Prepolymer Microcapsule with Second Crosslinker

An oil phase was prepared by mixing Magnitude fragrance (10%), caprylic/capric triglyceride (2.5%, NEOBEE® M-5) and polyisocyanate (0.25%, TAKENATE® D-110N). Cationic Guar (1.0%, available under the tradename N-HANCE® C261N, Ashland, USA) was subsequently dispersed into the oil phase under constant mixing. The oil phase mixture was heated to 60° C. at constant mixing and DABCO (0.015%) was added to catalyze the reaction for 10 minutes. Separately, an aqueous solution of gum Arabic (2.0%) was prepared and then emulsified with the oil phase at 6000 rpm for 3 minutes to form an emulsion. After allowing the emulsion to stir at 40° C. for 1 hour, the pH of the emulsion was gradually adjusted to 3.75 using 50% citric acid. A first aqueous crosslinking solution containing sucrose (1.0%) and sodium periodate (0.1%) was prepared and stirred at room temperature for 30 minutes. The first aqueous crosslinking solution was then added to the emulsion followed by the addition of the second crosslinking solution with the second crosslinker and the amount indicated in Table 25. The resulting emulsion maintained stirring at 40° C. for 4 hours (curing) to provide the microcapsule slurry (i.e., Microcapsules 53-55 respectively). The microcapsule slurry was then cooled and stirred at room temperature overnight. Component percentages in parentheses in Example 27 are based on total weight of the microcapsule slurry.

TABLE 25

| Microcapsule | 53 | 54 | 55 |
|---|---|---|---|
| Second Crosslinker | Tannic Acid | Glutaraldehyde | Glyoxal |
| Amount | 0.25% | 0.25% | 0.25% |

Note:
percentages are based on total weight of the microcapsule slurry (i.e., emulsion).

### Example 28: Collagen Prepolymer Microcapsule with Second Crosslinker

An oil phase was prepared by mixing Magnitude fragrance (10%), caprylic/capric triglyceride (2.5%, NEOBEE® M-5) and polyisocyanate (0.25%, TAKENATE® D-110N). Collagen (10.0%, available under the tradename BIOLLAGEN® SSE, Jiangsu JLand Biotech, China) was subsequently dispersed into the oil phase under constant mixing. The oil phase mixture was heated to 60° C. at constant mixing and DABCO (0.015%) was added to catalyze the reaction for 10 minutes. Separately, an aqueous solution of gum Arabic (1.0%) was prepared and then emulsified with the oil phase at 6000 rpm for 3 minutes to form an emulsion. After allowing the emulsion to stir at 40° C. for 1 hour, the pH of the emulsion was gradually adjusted to 3.5 using 50% citric acid. A first aqueous crosslinking solution containing sucrose (1.0%) and sodium periodate (0.1%) was prepared and stirred at room temperature for 30 minutes. The first aqueous crosslinking solution was then added to the emulsion followed by the addition of the second crosslinking solution with the second crosslinker and the amount indicated in Table 26. The resulting emulsion maintained stirring at 40° C. for 4 hours (curing) to provide the microcapsule slurry (i.e., Microcapsules 56-58 respectively). The microcapsule slurry was then cooled and stirred at room temperature overnight. Component percentages in parentheses in Example 28 are based on total weight of the microcapsule slurry.

TABLE 26

| Microcapsule | 56 | 57 | 58 |
|---|---|---|---|
| Second Crosslinker | Tannic Acid | Glutaraldehyde | Glyoxal |
| Amount | 0.25% | 0.25% | 0.25% |

Note:
percentages are based on total weight of the microcapsule slurry (i.e., emulsion).

### Example 29: Gum Arabic Prepolymer Microcapsule with Second Crosslinker

An oil phase was prepared by mixing Magnitude fragrance (10%), caprylic/capric triglyceride (2.5%, NEOBEE® M-5) and polyisocyanate (0.25%, TAKENATE® D-110N). Gum Arabic (4.0%) was subsequently dispersed into the oil phase under constant mixing. The oil phase mixture was heated with constant mixing and DABCO (0.015%) was added and heated at 60° C. for 10 minutes to catalyze the reaction. Separately, an aqueous solution of Chitosan (2.0%, commercially available under the tradename KiOsmetine™ P, KitoZyme, Belgium) and acetic acid (1.0%) was prepared and then emulsified with the oil phase at 6000 rpm for 3 minutes to form an emulsion. After allowing the emulsion to stir at 40° C. for 1 hour, the pH of the emulsion was gradually adjusted to 5.0 using 5% sodium hydroxide. A first aqueous crosslinking solution containing sucrose (1.0%) and sodium periodate (0.1%) was prepared and stirred at room temperature for 30 minutes. The first aqueous crosslinking solution was then added to the emulsion followed by the addition of the second crosslinking solution with the second crosslinker and the amount indicated in Table 27. The resulting emulsion maintained stirring at 40° C. for 4 hours (curing) to provide the microcapsule slurry (i.e., Microcapsules 59-61 respectively). The microcapsule slurry was then cooled and stirred at room temperature overnight. Component percentages in parentheses in Example 29 are based on total weight of the microcapsule slurry.

TABLE 27

| Microcapsule | 59 | 60 | 61 |
|---|---|---|---|
| Second Crosslinker | Tannic Acid | Glutaraldehyde | Glyoxal |
| Amount | 0.25% | 0.25% | 0.25% |

Note:
percentages are based on total weight of the microcapsule slurry (i.e., emulsion).

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination.

1. A biodegradable core-shell microcapsule comprising:
(a) a shell comprising the reaction product of an isocyanate-functionalized prepolymer with a crosslinker and optionally a polyelectrolyte under an aqueous condition; and
(b) a core comprising an active material;
wherein the isocyanate-functionalized prepolymer is the reaction product of a polyisocyanate with a biopolymer and/or an amphiphilic compound under an anhydrous condition,
the biopolymer is selected from the group consisting of gelatin, collagen, chitosan, modified guar, modified glucan, gum Arabic (gum acacia), modified gum Arabic (modified gum acacia), proteins, hydrolyzed proteins, fermented proteins, hydrophobin, enzymes, alginate, carrageenan, pectin, modified starch, modified cellulose, and mixtures thereof,
the amphiphilic compound is selected from the group consisting of partially neutralized acid esters, polyvinyl alcohol, glycolipids, fatty acids, saponins, quillaia extract, surfactant salts having carboxylate and/or linear alcohol groups, and mixtures thereof,
the microcapsule shell has a biodegradation rate of at least 20%, 30%, 40%, 50%, 60%, 65%, 70%, 75%, 80%, 85%, 90%, 95% or 98%, based on the weight of the microcapsule shell, within 60 days according to OECD301F, and
the microcapsule shell is substantially free of or free of a self-condensed polyisocyanate.

2. The biodegradable core-shell microcapsule of claim 1, wherein the crosslinker comprises a first crosslinker comprising an oxidized sugar and/or an enzyme, the oxidized sugar comprises aldehyde groups, and the enzyme is selected from the group consisting of transglutaminase, laccase, peroxidase, oxidase, amylase, transferase, and mixtures thereof.

3. The biodegradable core-shell microcapsule of claim 1, wherein the level of the self-condensed polyisocyanate is ≤10%, ≤5%, ≤3%, ≤1%, ≤0.5%, ≤0.1% or ≤0.05%, relative to the total weight of polyisocyanate used to form wall of the microcapsule.

4. The biodegradable core-shell microcapsule of claim 2, wherein the crosslinker further comprises a second crosslinker selected from the group consisting of tannic acid, hydrolyzed tannic acid, tannin, gallic acid, methyl gallate, ethyl gallate, glutaraldehyde, glyoxal, triethyl citrate, malondialdehyde, genipin, dopamine, phenols, polyphenols, polycarbodiimide, polyacid chlorides, tetraethoxysilane, enzymes, multivalent cations, and mixtures thereof.

5. The biodegradable core-shell microcapsule of claim 1, wherein the microcapsule shell is crosslinked with the crosslinker and the weight average molecular weight of crosslinked prepolymer and/or polyelectrolyte is at least 2 times, at least 4 times, or at least 10 times of the weight average molecular weight of non-crosslinked prepolymer and/or polyelectrolyte respectively, as determined by size exclusion chromatography.

6. The biodegradable core-shell microcapsule of claim 1, wherein the polyelectrolyte is selected from the group consisting of gelatin, collagen, chitosan, modified guar, modified glucan, gum Arabic (gum acacia), modified gum Arabic (modified gum acacia), proteins, hydrolyzed proteins, fermented proteins, hydrophobin, enzymes, partially neutralized citric acid ester, alginate, carrageenan, pectin, modified starch, modified cellulose, and mixtures thereof.

7. The biodegradable core-shell microcapsule of claim 1, wherein the active material comprises a fragrance, flavor, agricultural active, pesticide, insecticide, herbicide, fungicide, pharmaceutical active, nutraceutical active, animal nutrition active, food active, microbio active, malodor counteractant, and/or cosmetic active, preferably a fragrance, more preferably a fragrance comprising at least one, at least two, at least three, at least four, at least five, at least six, at least seven, at least eight, at least nine or at least ten High Performance fragrance ingredients selected from the group consisting of Ultra High-Impact fragrance ingredients as listed in Table 1 and High-Impact fragrance ingredients as listed in Table 2, and optionally at least one additional fragrance ingredient.

8. A process for preparing a biodegradable core-shell microcapsule, comprising:
(a) reacting, under an anhydrous condition, a polyisocyanate with a biopolymer and/or an amphiphilic compound, preferably in the presence of a catalyst, to form an isocyanate-functionalized prepolymer, preferably the polyisocyanate is dissolved in a solution comprising a solvent and/or an active material;
(b) emulsifying the isocyanate-functionalized prepolymer with an aqueous solution to form an emulsion, preferably said aqueous solution comprises a polyelectrolyte;
(c) crosslinking the isocyanate-functionalized prepolymer and optionally the polyelectrolyte with a first crosslinker to form the biodegradable core-shell microcapsule, wherein the first crosslinker comprises an oxidized sugar comprising aldehyde groups and/or an enzyme selected from the group consisting of transglutaminase, laccase, peroxidase, oxidase, amylase, transferase, and mixtures thereof;
(d) optionally further crosslinking the microcapsule shell with a second crosslinker selected from the group consisting of tannic acid, hydrolyzed tannic acid, tannin, gallic acid, methyl gallate, ethyl gallate, glutaraldehyde, glyoxal, triethyl citrate, malondialdehyde, genipin, dopamine, phenols, polyphenols, polycarbodiimide, polyacid chlorides, tetraethoxysilane, enzymes, multivalent cations, and mixtures thereof; and
(e) optionally curing the microcapsule shell at a temperature ranging from 5° C. to 150° C. and at a pH ranging from 2 to 11;
wherein the microcapsule shell has a biodegradation rate of at least 20%, 30%, 40%, 50%, 60%, 65%, 70%, 75%, 80%, 85%, 90%, 95% or 98%, based on the weight of the microcapsule shell, within 60 days according to OECD301F, and
the microcapsule shell is substantially free of or free of a self-condensed polyisocyanate.

9. The process of claim 8, wherein the biopolymer is selected from the group consisting of gelatin, collagen, chitosan, modified guar, modified glucan, gum Arabic (gum acacia), modified gum Arabic (modified gum acacia), proteins, hydrolyzed proteins, fermented proteins, hydrophobin, enzymes, alginate, carrageenan, pectin, modified starch, modified cellulose, and mixtures thereof, and the amphiphilic compound is selected from the group consisting of partially neutralized acid esters, polyvinyl alcohol, glycolipids, fatty acids, saponins, quillaia extract, surfactant salts having carboxylate and/or linear alcohol groups, and mixtures thereof.

10. The process of claim 8, wherein the polyelectrolyte is selected from the group consisting of gelatin, collagen, chitosan, modified guar, modified glucan, gum Arabic (gum acacia), modified gum Arabic (modified gum acacia), proteins, hydrolyzed proteins, fermented proteins, hydrophobin, enzymes, partially neutralized citric acid ester, alginate, carrageenan, pectin, modified starch, modified cellulose, and mixtures thereof.

11. The process of claim 8, wherein the first crosslinker comprises an oxidized sugar, and the sugar is selected from the group consisting of glucose, glucosamine, sucrose, maltose, lactose, maltodextrin, cyclodextrin, polysaccharide, hydrolyzed polysaccharide, and mixtures thereof.

12. The process of claim 8, wherein the active material comprises a fragrance, flavor, agricultural active, pesticide, insecticide, herbicide, fungicide, pharmaceutical active, nutraceutical active, animal nutrition active, food active, microbio active, malodor counteractant, and/or cosmetic active, preferably a fragrance, more preferably a fragrance comprising at least one, at least two, at least three, at least four, at least five, at least six, at least seven, at least eight, at least nine or at least ten High Performance fragrance ingredients selected from the group consisting of Ultra High-Impact fragrance ingredients as listed in Table 1 and High-Impact fragrance ingredients as listed in Table 2, and optionally at least one additional fragrance ingredient.

13. A biodegradable core-shell microcapsule obtainable by the process of claim 8.

14. A consumer product comprising the biodegradable core-shell microcapsule of claim 1, preferably the consumer product is a fabric softener, fabric conditioner, detergent, scent booster, fabric refresher spray, body wash, body soap, shampoo, hair conditioner, body spray, hair refresher spray, hair dye, hair moisturizer, skin moisturizer, hair treatment, skin treatment, antiperspirant, deodorant, insect repellant, candle, surface cleaner, bathroom cleaner, bleach, cat litter, refresher spray, pesticide, insecticide, herbicide, fungicide, or paint.

15. A process for making an isocyanate-functionalized prepolymer, comprising: reacting, under an anhydrous condition, a polyisocyanate with a biopolymer and/or an amphiphilic compound, preferably in the presence of a catalyst, to form an isocyanate-functionalized prepolymer,
wherein the polyisocyanate is dissolved in a solution comprising a solvent and/or an active material,
the biopolymer is selected from the group consisting of gelatin, collagen, chitosan, modified guar, modified glucan, gum Arabic (gum acacia), modified gum Arabic (modified gum acacia), proteins, hydrolyzed proteins, fermented proteins, hydrophobin, enzymes, alginate, carrageenan, pectin, modified starch, modified cellulose, and mixtures thereof, and
the amphiphilic compound is selected from the group consisting of partially neutralized acid esters, polyvinyl alcohol, glycolipids, fatty acids, saponins, quillaia extract, surfactant salts having carboxylate and/or linear alcohol groups, and mixtures thereof.

16. An isocyanate-functionalized prepolymer obtainable by the process of claim 15, preferably the isocyanate-functionalized prepolymer has a biodegradation rate of at least 20%, 30%, 40%, 50%, 60%, 65%, 70%, 75%, 80%, 85%, 90%, 95% or 98%, based on the weight of the isocyanate-functionalized prepolymer, within 60 days according to OECD301F.
